(19) United States
(12) Patent Application Publication
(10) Pub. No.: US 2018/0344715 A1
(43) Pub. Date: Dec. 6, 2018

(54) WNT/BETA-CATENIN SIGNAL TRANSDUCTION INHIBITORS AND THEIR USE IN TREATMENT OR PREVENTION OF DISEASES AND CONDITIONS LINKED WITH SAID TRANSDUCTION

(71) Applicant: BERGEN TEKNOLOGIOVERFORING AS, BERGEN (NO)

(72) Inventors: YI QU, BERGEN (NO); ANNE MARGRETE OYAN, BERGEN (NO); KARL-HENNING KALLAND, BERGEN (NO); XISONG KE, BERGEN (NO)

(21) Appl. No.: 15/771,496

(22) PCT Filed: Oct. 28, 2016
(86) PCT No.: PCT/EP2016/076171

§ 371 Date: Apr. 27, 2018

(30) Foreign Application Priority Data
Oct. 30, 2015 (GB) 1519258.6

(51) Int. Cl. A61K 31/4439; A61K 31/506; A61K 31/337; A61K 31/4745; A61P 35/00; G01N 33/574

(52) U.S. Cl. CPC A61K 31/4439; A61K 31/506; G01N 33/574; A61K 31/4745; A61P 35/00; A61K 31/337

(57) ABSTRACT

In the invention provides a WNT/β-catenin signal transduction inhibitor selected from (i) axitinib, (ii) pazopanib, (iii) orlistat, (iv) topotecan, (v) pharmaceutically effective substitution derivatives thereof, or (vi) pharmaceutically acceptable salts, or solvates or hydrates thereof, diastereoisomers, tautomers, enantiomers, and prodrugs and active metabolites thereof, for use in the treatment or prevention of a disease or condition in which WNT/β-catenin signal transduction is a contributing factor and for use in a method for the immunotherapy of a hyperproliferative or neoplastic disease or condition in a subject in which DCs are administered to the subject. The invention further provides an in vitromethod for diagnosing WNT/β-catenin dependent cancers, said method comprising (i) contacting a sample of cells from a test cancer with one or more of the WNT/β-catenin signal transduction inhibitors disclosed herein, and (ii) assessing the effects of said inhibitor on said sample.

The present invention provides newly identified WNT/β-catenin signal transduction inhibitors and the use thereof in the treatment or prevention of diseases and conditions in which WNT/β-catenin signal transduction is a contributing factor. It is known that certain cancers may be dependent on WNT/β-catenin signal transduction during their establishment, maintenance, growth and subsequent spread (metastasis) or may evade the host immune system through WNT/β-catenin dependent processes. Inhibitors of such oncogenic and immune evasive signalling may be used in the treatment or prevention of such cancers. It is also becoming clear that certain immune and inflammatory diseases and conditions are mediated by WNT/β-catenin signal transduction, e.g. autoimmune diseases, transplant rejection, inflammatory bowel disease, multiple sclerosis, chronic microbial infection. Inhibitors of such signalling in the immune and inflammatory systems may be used in the treatment or prevention of such diseases or conditions. It is also becoming clear that certain disorders and dysfunctions in the metabolism of carbohydrates are mediated by WNT/β-catenin signal transduction, e.g. insulin resistance, diabetes type 2, obesity and metabolic syndrome. Inhibitors of such signalling in the context of the metabolism of carbohydrates may be used in the treatment or prevention of such disorders and dysfunctions. It is also becoming clear that WNT/β-catenin signal transduction plays a role in the process of wound healing, in particular cutaneous wound healing. Manipulation of such signalling in the context of wounds may be used in the treatment of chronic wounds by promoting the healing process. It has now been found surprisingly that axitinib, pazopanib, orlistat and topotecan may function as WNT/β-catenin signal transduction inhibitors and as such may be used in the above described therapies. The invention further provides an in vitro method for diagnosing WNT/β-catenin dependent cancers in which the susceptibility of a sample cell from a target cancer to one or more of the newly identified WNT/β-catenin signal transduction inhibitors is assessed.

The WNT/β-catenin signal transduction pathway is a well characterised intracellular signalling pathway in metazoan animal cells and is the subject of many review articles, e.g. McDonald et al. 2009 Dev. Cell 17: 9-26; Clevers and Nusse, 2012, Cell 149: 1192-1205; Yokonama N N et al., 2014, Am. J. Clin. Exp. Urol. 2: 27-44; and Takebe N et al., 2015, Nat. Rev. Clin. Oncol. 12:445-64, the contents of which are incorporated herein by reference.

The WNT/β-catenin signal transduction pathway is based on the control of the rate of β-catenin turnover in the cytoplasm of a cell. β-catenin is a transcriptional co-activator of nuclear transcription factors that belong to the TCF/LEF (T-cell factor/lymphoid enhancing factor) family and as such reduced turnover and subsequent accumulation of β-catenin in the cytoplasm eventually results in translocation of β-catenin to the nucleus where it exerts its inherent transcriptional co-activator effects. β-catenin turnover is controlled by an intracellular destruction complex comprising, inter alia, axin, the tumor suppressor adenomatosis polyposis coli (APC), protein phosphatase 2A (PP2A), glycogen synthase kinase 3 (GSK3) and casein kinase 1α (CK1α). This complex promotes constitutive degradation of β-catenin by phosphorylating β-catenin with GSK3 and CK1α and thereby targeting it for ubiquitination and proetosomal degradation. This continual elimination of β-catenin prevents effective levels of β-catenin accumulating in the nucleus, and WNT target genes are thereby repressed by the DNA-bound T cell factor/lymphoid enhancer factor (TCF/LEF) family of proteins.

Upon activation of the WNT/β-catenin signal transduction pathway, by the binding of a WNT protein to the extracellular domain of a member of the Frizzled family of G-protein coupled receptors in complex with the co-receptor lipoprotein receptor-related protein (LRP)-5/6, the function of the destruction complex becomes disrupted. This involves a process by which the axin and the rest of the destruction complex translocates to the activated receptor complex where it binds to the cytoplasmic tail of LRP-5/6. Axin subsequently becomes de-phosphorylated and its stability is decreased, lowering its levels in the destruction complex. Disheveled (Dsh) then becomes activated via phosphorylation by CK1α and in this form inhibits the GSK3 activity of the destruction complex. This suppresses β-catenin degradation thus allowing β-catenin to accumulate and then to translocate to the nucleus. In the nucleus β-catenin induces cellular responses via gene transcription alongside the TCF/LEF transcription factors.

WNT/β-catenin signal transduction is one of the fundamental mechanisms that direct cell proliferation, cell polarity, and cell fate determination during embryonic development and tissue homeostasis. Consequently WNT/β-catenin signal transduction has been associated with birth defects, cancer, and other hyperproliferative diseases.

Mutations in the components of the pathway, e.g. the APC and CTNNB1 (8-catenin) genes, which directly give rise to overactivity in the pathway and therefore oncogenic signalling are known. Such mutations include those which suppress β-catenin turnover, increase β-catenin expression, increase translocation of β-catenin in the nucleus, increase the intrinsic activity of β-catenin or other stimulatory components of the pathway or decrease the intrinsic activity of inhibitory components of the pathway. In susceptible cell types this overactivity can lead to the establishment of a tumour, its progression and potentially its metastasis. It has also been recognised that WNT/β-catenin signal transduction, but not necessarily overactivity, is an essential requirement for the growth of certain tumours and their metastasis, e.g. by permitting the cells of the tumour to metabolise glucose at a sufficient rate to permit growth of the tumour.

More recently WNT/β-catenin signal transduction has been recognised as a contributing factor in the aetiology of certain immune or inflammatory diseases and, through effects on immune cells, may contribute to the pathology of certain tumours, e.g. the establishment, progression and metastasis of certain tumours (Swafford, D. and Manicassamy, S., 2015, Discovery Medicine No 105; Fu, C. et al., 2015, PNAS, Vol 112: 2823-2828 and Spranger S. et al., Nature, 2015, Vol 523: 231-235, the contents of which are incorporated herein by reference).

For instance, it has been found that WNT/β-catenin signal transduction in effector T cells and/or T_{reg} cells is causatively linked with the imprinting of proinflammatory properties (Swafford and Manicassamy, supra; Shilpa, K., 2014, Science Translational Medicine, Vol. 6 (225), 225-228). Such effects have been shown to contribute to chronic inflammation of the intestine and the colon, such as in IBD and ulcerative colitis in particular, and the subsequent establishment and development of intestinal and colon cancers. A role for such effects in autoimmune disease and transplant rejection has also been proposed (Swafford and Manicassamy, supra)

In further examples, WNT/β-catenin signal transduction may affect tolerogenic signalling in dendritic cells (DCs) promoting the switch from a tolerogenic state to a proinflammatory state. Tolerogenic DCs regulate immunological responses to self-antigens and other innocuous antigens and thus breakdown in this DC mediated immunological tolerance is predicted to give rise to autoimmune disease, allograft transplant rejection and susceptibility to allergies.

Certain tumours have been shown to be able to evade the host immune system and thereby prevent their eradication by effector T cells (e.g. cytotoxic T lymphocytes). Certain mechanisms by which such tumours combat anti-tumour T cells responses have been shown to involve WNT/β-catenin signal transduction in the tumour cells and/or DCs (Swafford and Manicassamy, supra; Fu, C., et al, 2015, PNAS, Vol 112(9), 2823-2828; Spranger, S., et al, 2015, Nature, 523, 231-235). Intrinsic WNT/β-catenin signal transduction in tumour cells results in T cell exclusion from the tumour. WNT/β-catenin signal transduction in DCs during the priming phase suppresses CD8⁺ T cell (cytotoxic T lymphocyte) anti-tumour immunity by inhibition of the cross-priming reactions. WNT/β-catenin signal transduction in DCs is associated with tolerogenic signalling in DCs and thus a reduced anti-tumour immune response. WNT/β-catenin signal transduction in cancer cells and DCs may also play a role in the promotion of T_{reg} cell activity by tumours, which further suppresses the anti-tumour immune response.

WNT/β-catenin signal transduction has also been recognised recently as a contributing factor in certain disorders and dysfunctions in the metabolism of carbohydrates. In the context of diabetes mellitus type 2, gain of function mutations in certain β-catenin activated transcription factors have shown to be a risk factor for diabetes mellitus type 2 and thus insulin resistance, metabolic syndrome and obesity (Clevers and Nusse, supra).

WNT/β-catenin signal transduction has also been recognised recently as playing a role in the process of wound healing, in particular cutaneous wound healing. It has been found that the migration and proliferation of keratinocytes and fibroblasts may be regulated by WNT/β-catenin signal transduction and that the other key signalling pathways, e.g. TNF-β mediated pathways, may be influenced by WNT/β-catenin signal transduction (Stojadinovic, O., et al, 2005, Am J Pathol. Vol 167(1), 59-69; Cheon, S., 2006, FASEB J, Vol 20(6), 692-701). Moreover the process of wound healing has an inflammatory component and as such the role WNT/β-catenin signal transduction plays in wound healing may also be effected through regulation of immune cells in the wound and surrounding tissues.

WNT/β-catenin signal transduction has also been recognised as a contributing factor in high bone mass disorders and sclerosteosis where it positively regulates osteoblast proliferation (Clevers and Nusse, supra).

In view of the foregoing it will be seen that WNT/β-catenin signal transduction represents a key target for therapeutic intervention and, in particular, inhibition of WNT/β-catenin signal transduction in the above described contexts would provide therapies for the above mentioned diseases, conditions, disorders or dysfunctions. WNT/β-catenin signal transduction inhibitors are known (e.g. Clevers and Nusse, supra, Table 2; WO2013/105022) but further examples are needed, especially examples that are known to be safe for human use.

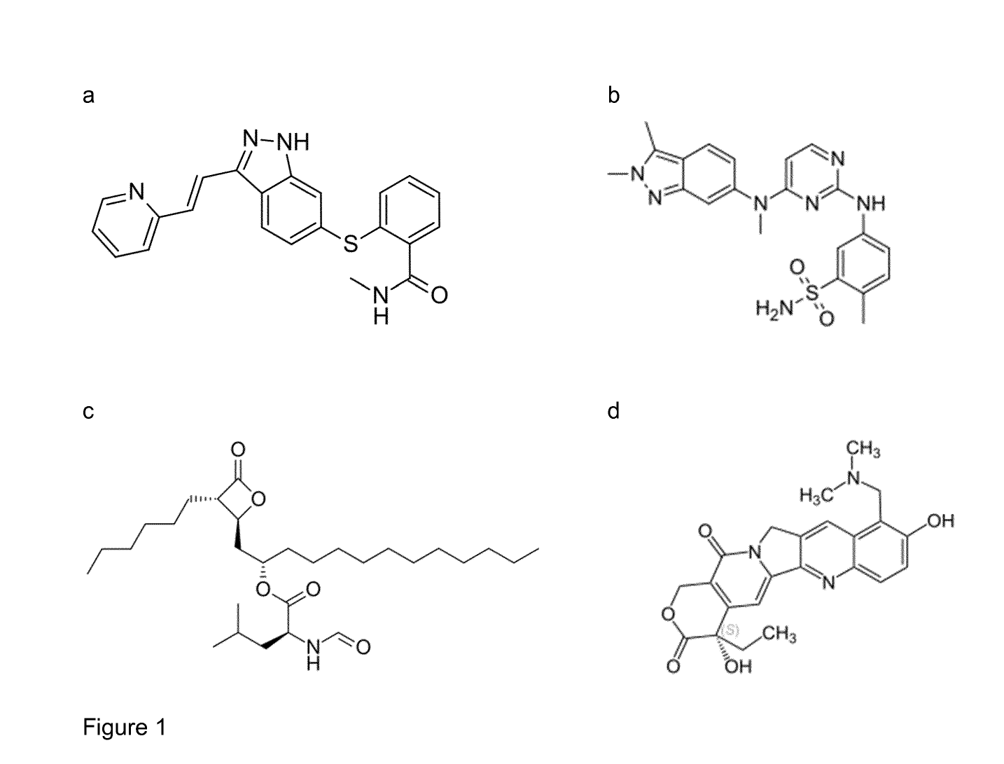

Axitinib (AG013736; Inlyta™, N-Methyl-2-[[3-[(E)-2-pyridin-2-ylethenyl]-1H-indazol-6-yl]sulfanyl]benzamide; FIG. 1a) is an orally administered small molecule tyrosine kinase inhibitor approved for the second-line treatment of patients with advanced renal cell carcinoma (RCC). Its primary mechanism of action is thought to be inhibition of vascular endothelial growth factor receptors 1 to 3, c-KIT and PDGFR, which enables it to inhibit angiogenesis

Pazopanib (Votrient™; 5-[[4-[(2,3-Dimethyl-2H-indazol-6-yl)methylamino]-2-pyrimidinyl]amino]-2-methylbenzolsulfonamide; FIG. 1b) is an orally administered tyrosine kinase inhibitor approved for the first-line and second-line treatment of advanced renal cell carcinoma and advanced soft tissue sarcomas. Its primary mechanism of action is through the inhibition of c-Kit and VEGF, PDGF, and FGF receptors on cancer cells, vascular endothelial cells and pericytes, which halts the proliferation of tumour cells and the development of tumour blood vessels.

Orlistat (Xenical™; Alli™; tetrahydrolipstatin; (S)-((S)-1-((2S,3S)-3-Hexyl-4-oxooxetan-2-yl)tridecan-2-yl) 2-formamido-4-methylpentanoate; FIG. 1c) is an orally administered lipase inhibitor approved for the treatment of obesity. Its primary mechanism is to prevent the absorption of dietary fats thereby reducing caloric intake. There is some evidence that orlistat is able to inhibit fatty acid synthase, an enzyme overexpressed in certain tumours.

Topotecan (Hycamtin™; (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride; FIG. 1d) is an orally administered inhibitor of topoisomerase I approved for the treatment of small-cell lung cancer, cervical cancer and ovarian cancer. Its primary mechanism is to inhibit DNA replication and promote apoptosis.

It has now been found surprisingly that axitinib, pazopanib, orlistat and topotecan, all of which are approved for use in humans to treat various clinical ailments, are able to function as WNT/β-catenin signal transduction inhibitors.

Thus, in a first aspect the invention provides a method for the treatment or prevention of a disease or condition in which WNT/β-catenin signal transduction is a contributing factor, said method comprising administering to a subject in need thereof one or more WNT/β-catenin signal transduction inhibitors selected from
(i) axitinib,
(ii) pazopanib,
(iii) orlistat,
(iv) topotecan,
(v) pharmaceutically effective substitution derivatives thereof wherein one or more hydrogen groups are substituted with SR′ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, or
(vi) pharmaceutically acceptable salts, or solvates or hydrates thereof, diastereoisomers, tautomers, enantiomers, and prodrugs and active metabolites thereof.

Expressed alternatively the invention provides a WNT/β-catenin signal transduction inhibitor selected from
(i) axitinib,
(ii) pazopanib,
(iii) orlistat,
(iv) topotecan,
(v) pharmaceutically effective substitution derivatives thereof wherein one or more hydrogen groups are substituted with SR′ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, or
(vi) pharmaceutically acceptable salts, or solvates or hydrates thereof, diastereoisomers, tautomers, enantiomers, and prodrugs and active metabolites thereof,
   for use in the treatment or prevention of a disease or condition in which WNT/6-catenin signal transduction is a contributing factor.

Expressed alternatively the invention provides the use of a WNT/β-catenin signal transduction inhibitor selected from
(i) axitinib,
(ii) pazopanib,
(iii) orlistat,
(iv) topotecan,
(v) pharmaceutically effective substitution derivatives thereof wherein one or more hydrogen groups are substituted with SR′ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, or
(vi) pharmaceutically acceptable salts, or solvates or hydrates thereof, diastereoisomers, tautomers, enantiomers, and prodrugs and active metabolites thereof,
   in the manufacture of a medicament for use in the treatment or prevention of a disease or condition in which WNT/β-catenin signal transduction is a contributing factor.

In the following discussion references to axitinib, pazopanib, orlistat and topotecan include pharmaceutically effective substitution derivatives thereof wherein one or more hydrogen groups are substituted with SR′ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, and pharmaceutically acceptable salts, or solvates or hydrates thereof, diastereoisomers, tautomers, enantiomers, and prodrugs thereof, unless context specifically dictates otherwise.

The term “pharmaceutically acceptable salt” refers to salts of the WNT/β-catenin signal transduction inhibitors of use in the invention prepared from pharmaceutically acceptable bases or acids, including inorganic or organic bases and inorganic or organic acids. Salts in the solid form can exist in more than one crystal structure and can also be in the form of hydrates and polyhydrates.

Salts derived from inorganic bases include aluminum, ammonium, calcium, copper, ferric, ferrous, lithium, magnesium, manganic, manganous, potassium, sodium, and zinc salts, and the like. The ammonium, calcium, magnesium, potassium, and sodium salts, in particular, can be preferred for some pharmaceutical formulations. Salts derived from pharmaceutically acceptable organic bases include salts of primary, secondary and tertiary amines, substituted amines, including naturally occurring substituted amines, cyclic amines, arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine, and the like.

When the WNT/β-catenin signal transduction inhibitors of use in the invention to be formulated is basic, salts can be prepared from pharmaceutically acceptable acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acid, and the like. Illustrative pharmaceutically acceptable acids include citric, hydrobromic, hydrochloric, maleic, phosphoric, sulfuric, and tartaric acids. Suitable pharmaceutically acceptable salts of the WNT/β-catenin signal transduction inhibitors of use in the invention include, but are not limited to, the mesylate, maleate, fumarate, tartrate, hydrochloride, hydrobromide, esylate, p-toluenesulfonate, benzoate, acetate, phosphate, and sulfate salts, preferably the hydrochloride salts.

Prodrugs are compounds that are converted to the therapeutically active compounds of use in the invention as they are being administered to a subject or after they have been administered to a subject. Active metabolites of the WNT/β-catenin signal transduction inhibitors of use in the invention are breakdown products retaining at least about 50%, e.g. at least about 75%, 85%, 90% or 95%, of the WNT/β-catenin signal transduction inhibitor activity of the non-metabolised inhibitor.

The diseases or conditions treatable in accordance with the invention are diseases or conditions (or disorders or dysfunctions, which terms are used interchangeably herein), specifically clinical, e.g. pathological, diseases or conditions, in which WNT/β-catenin signal transduction is a factor which contributes to, or which underlies, any aspect of the pathology of the disease or condition, e.g. the establishment and/or progression of the disease or condition. In other words WNT/β-catenin signal transduction is an effector of such diseases and conditions. Thus WNT/β-catenin signal transduction may initiate the disease process or a new phase thereof, may be involved in the development of a disease or phase thereof that has already been established and/or may be involved in maintaining or consolidating a disease or a phase thereof. Thus, the diseases or conditions treated or prevented in accordance with the invention may be diseases or conditions which are mediated by, promoted by, caused by, dependent on, associated with, or which involve or result from WNT/β-catenin signal transduction.

In accordance with the invention references to “WNT/β-catenin signal transduction” are references to active WNT/β-catenin signal transduction, i.e. a state of active signalling through the pathway (which may be a normal level of signalling or a level which is increased compared to normal) and should not be construed as references to states in which there is an absence, lack of, or insufficient signalling. As such, the diseases or conditions treated or prevented in accordance with the invention may also be diseases or conditions which are capable of being alleviated or prevented by reducing (inhibiting, suppressing, abrogating, deactivating) WNT/β-catenin signal transduction (e.g. signal transduction which is reduced from normal levels or levels which are increased compared to normal).

In certain embodiments the diseases or conditions treated or prevented in accordance with the invention may be diseases or conditions in which WNT/β-catenin signal transduction in a neoplastic cell is a contributing factor. References to a neoplastic cell or a neoplasm include any or all of malignant, pre-malignant and non-malignant (benign) neoplastic entities. The term therefore encompasses, inter alia, cancer cells (cancers), tumour cells (tumours), malignant cells (malignancies), sarcoma cells (sarcomas), carcinoma cells (carciomas), germinoma cells (germinomas), lymphoma cells (lymphonas) and leukaemia cells (leukaemias), blastoma cells (blastomas), papilloma cells (papillomas) and adenoma cells (adenomas). In certain embodiments the neoplastic cell is a cancer cell or malignant or premalignant tumour cell, e.g. a sarcoma cell, carcinoma cell, germinoma cell, blastoma cell, lymphoma cell or a leukaemia cell.

In these embodiments the method of the invention may be considered to be a method for the treatment or prevention of a hyperproliferative or neoplastic disease or condition, e.g. those recited above, in which WNT/β-catenin signal transduction in a neoplastic cell is a contributing factor.

In more specific embodiments the method of the invention may be considered to be a method for the treatment or prevention of a cancer or a malignant or premalignant tumour in which WNT/β-catenin signal transduction in a neoplastic cell is a contributing factor.

Expressed differently, the method of the invention may be considered to be a method for the treatment or prevention of a WNT/β-catenin dependent cancer or malignant or premalignant tumour, i.e. a cancer or malignant or premalignant tumour which is mediated by, promoted by, caused by, associated with, or which involves or results from WNT/β-catenin signal transduction in a neoplastic cell.

In certain embodiments there may be increased WNT/β-catenin signal transduction compared to normal (i.e. normal levels of WNT/β-catenin signal transduction in a non-transformed version of the target neoplastic cell). This may be referred to as overactivity in the WNT/β-catenin signal transduction pathway. This may also be expressed as WNT/β-catenin oncogenic signalling and as such the method of the invention may be considered to be a method for the treatment or prevention of a cancer or malignant or premalignant tumour in which WNT/β-catenin oncogenic signalling in a neoplastic cell is a contributing factor.

Increased WNT/β-catenin signal transduction may arise by virtue of gain of function mutations in the positive (agonistic) regulators of WNT/β-catenin signal transduction (effectors), e.g. the CTNNB1 (β-catenin), WNT, FZD (Frizzled), LRP (co-receptor lipoprotein receptor-related protein) and TCF genes, and/or loss of function mutations in negative (antagonistic) regulators of WNT/β-catenin signal transduction (suppressors), e.g. the APC and Axin genes. In other embodiments increased WNT/β-catenin signal transduction may arise from a loss of, or reduction in, parallel functionally antagonistic signalling (tumour suppressor signalling) or from an increase in agonistic signalling which feeds into the WNT/β-catenin signal transduction system.

In preferred embodiments the cancer or malignant or premalignant tumour treated or prevented by the methods of the invention are cancers or malignant or premalignant tumours carrying one or more agonistic mutant forms of the components of the WNT/β-catenin signal transduction pathway, preferably a gain of function mutation in one or more of a CTNNB1, WNT, FZD or TCF gene, and/or a loss of function mutation in one or more of an APC or an Axin gene.

Loss of asymmetric cell division is believed to be a critical event in cancer initiation and progression. It is shown in the Examples that asymmetric cell division is negatively mediated by WNT/β-catenin signal transduction and that axitinib (and by extension the other WNT/β-catenin signal transduction inhibitors of use in the invention) can re-establish asymmetric cell division. Thus, the method of the invention may be considered to be a method for the treatment or prevention of a cancer or malignant or premalignant tumour displaying WNT/β-catenin dependent loss of asymmetric cell division, i.e. a loss of asymmetric cell division which is mediated by, promoted by, caused by, associated with, or which involves or results from WNT/β-catenin signal transduction in a neoplastic cell. In these embodiments the method of the invention may, at least partially, re-establish asymmetric cell division in the cells displaying WNT/β-catenin dependent loss of asymmetric cell division. Expressed numerically at least about 10%, e.g. at least about 25%, 50%, 75% or 90%, of cells displaying essentially symmetric cell divisional prior to treatment are re-established in asymmetric cell division.

Asymmetric cell division may be measured by any convenient means. As shown in the Examples immunocytochemistry based approaches may be used.

In these various embodiments the hyperproliferative or neoplastic disease or condition may be selected from colorectal cancer (also known as colon cancer, rectal cancer or bowel cancer), prostate cancer, kidney (renal) cancer (e.g. Wilm's tumour), pancreatic cancer, testicular cancer, skin cancer (e.g. melanoma and non-melanoma (e.g. basal-cell cancer, squamous-cell cancer)), breast cancer, ovarian cancer, stomach (gastric) cancer, intestinal cancer (e.g. duodenal cancer, ileal cancer, jejunal cancer, small intestine cancer), liver (hepatic) cancer, lung (pulmonary) cancer, oesophageal cancer, oral cancer, throat cancer, brain cancer (e.g. glioblastoma, medulloblastoma), adrenal cancer (e.g. adrenocortical cancer), thyroid cancer (e.g. anaplastic thyroid carcinoma), uterine cancer (e.g. uterine carcinosarcoma), haematological cancer (also known as the haematological malignancies) (e.g. haematopoietic and lymphoid cancer malignancies, e.g. leukemia, lymphoma and myeloma), including metastatic forms thereof, and non-malignant neoplasm or tumour in these anatomical sites (e.g. colorectal polyps, pilomatrixoma, hemangioma, osteoma, chondroma, lipoma, fibroma, lymphangioma, leiomyoma, rhabdomyoma, astrocytoma, meningioma, ganglioneuroma, papilloma, adenoma).

In preferred embodiments the hyperproliferative or neoplastic disease or condition is selected from the abovementioned cancers. Colorectal cancer, prostate cancer, renal cancer and pancreatic cancer are of note and colorectal cancer and prostate cancer are of particular note.

It is now known that for a neoplastic disease to become established, to progress and/or to spread (e.g. metastasise) the neoplastic cells must evade the host immune system to at least some degree (referred to as tumour-mediated immune suppression, or cancer-mediated immune suppression) otherwise the host's immune system would eliminate the anomalous cells (referred to as anti-tumour immunity or anti-cancer immunity). It is also known that certain cancers are dependent on inflammatory mediators (e.g. cytokines) expressed by immune cells in order to become established, to progress and/or to spread (e.g. metastasise). These processes are believed to be, at least in part, mediated by WNT/β-catenin signal transduction in immune cells, e.g. those recited below, in particular DCs and macrophages.

Thus, in still further embodiments the method of the invention is a method for the treatment or prevention of a hyperproliferative or neoplastic disease or condition in which WNT/β-catenin signal transduction in an immune cell, e.g. those recited below, is a contributing factor. The hyperproliferative or neoplastic disease or condition may be any of those recited above, but not necessarily with limitation to those in which WNT/β-catenin signal transduction in a neoplastic cell is a contributing factor. Such treatments may be used together with a cancer immunotherapy.

By “use together” it is meant that the WNT/β-catenin signal transduction inhibitor of use in the invention may conveniently be administered before, simultaneously with or following the cancer immunotherapy. Conveniently the inhibitor is applied at substantially the same time as the cancer immunotherapy or afterwards. In other embodiments the inhibitor may conveniently be applied or administered before the cancer immunotherapy. The cancer immunotherapy can be given (e.g. administered or delivered) repeatedly at time points appropriate for the agent(s) used. The skilled person is able to devise a suitable dosage regimen. In long term treatments the inhibitor and the cancer immunotherapy can be used repeatedly. The inhibitor can be applied as frequently as the cancer immunotherapy, or more or less frequently. In these embodiments the method may be considered a method for improving or enhancing the effectiveness (or efficacy) of a cancer immunotherapy, said method comprising using one or more of the WNT/β-catenin signal transduction inhibitors described herein together with the cancer immunotherapy. Said improvement or enhancement is measured relative to effectiveness/efficacy in the absence of said inhibitor.

In the particular context of immune suppression by neoplastic cells it is DCs which have emerged as key immune cell mediators of this process. In one instance WNT/β-catenin signal transduction in DCs during the priming phase suppresses CD8⁺ T cell (cytotoxic T lymphocytes) anti-tumour immunity by inhibition of the cross-priming reactions. WNT/β-catenin signal transduction in DCs is also associated with tolerogenic signalling and thus a reduced anti-tumour immune response. On the other hand, intrinsic WNT/β-catenin signal transduction in cancer cells results in T cell exclusion from the tumour. WNT/β-catenin signal transduction in cancer cells and DCs may also play a role in the promotion of T_{reg} cell activity by cancers, which further suppresses the anti-tumour immune response. Inhibition of WNT/β-catenin signal transduction in these cells would combat (e.g. reduce, abrogate, reverse or eliminate) these pro-cancer outcomes.

Thus in still further embodiments the method of the invention is a method for the treatment of a hyperproliferative or neoplastic disease or condition in which WNT/β-catenin signal transduction in an immune cell, e.g. a DC, is mediating the suppression of the subject's anti-cancer immune response. In these embodiments treatment is effected by combating (e.g. reducing, abrogating, reversing or eliminating) the above described suppression mechanisms with the WNT/β-catenin signal transduction inhibitors of use in accordance with the invention. The hyperproliferative or neoplastic disease or condition may be any of those recited above, but not necessarily with limitation to those in which WNT/β-catenin signal transduction in a neoplastic cell is a contributing factor.

Expressed differently the method of the invention is a method for combating (e.g. reducing, abrogating, reversing or eliminating) cancer mediated immune suppression and/or augmenting (e.g. increasing, enhancing) anti-cancer immunity, specifically by combating the above described WNT/β-catenin signal transduction mediated mechanisms.

DC based cancer immunotherapy (DC based cancer vaccination) is emerging as a promising therapy for hyperproliferative or neoplastic diseases or conditions. The therapy is based on the principle that anti-cancer immunity is driven by DCs and by administering DCs (usually autologous DCs) to a subject with a hyperproliferative or neoplastic disease or condition the subject's immunity against said diseases and condition may be augmented. Some approaches involve administering immature DCs, i.e. DCs that have not been exposed to, or begun to display, cancer antigens. Exposure and subsequent display of cancer antigens takes place upon administration of the immature DCs to the subject, and more specifically the neoplasm or the remnants thereof following ablation (e.g. cryoablation) or other such destruction. Other approaches involve administering mature or maturing DCs which have been exposed to cancer antigens, either in vivo or in vitro.

Given the above described role of intrinsic DC WNT/β-catenin signal transduction in inhibiting the priming of effector T cells, in the promotion of tolerogenic DC phenotypes and in the promotion of T_{reg} cells, the WNT/β-catenin signal transduction inhibitors of use in the invention may therefore find specific application in the context of DC based cancer immunotherapy.

Thus a further aspect of the invention provides a method for the immunotherapy of a hyperproliferative or neoplastic disease or condition in a subject in which DCs are administered to the subject, said method comprising administering an effective amount of one or more of the WNT/β-catenin signal transduction inhibitors of use in the invention, i.e. axitinib, pazopanib, orlistat and topotecan, preferably pazopanib, orlistat and topotecan, to a subject at the same time as, or substantially the same time as, or prior to, or after said subject receives the DCs.

In a particular embodiment of this aspect of the invention the method for the immunotherapy of a hyperproliferative or neoplastic disease or condition in a subject in which DCs are administered to the subject comprises providing a sample of DCs in vitro, and either
(a) administering said DCs to the subject at the same time as, or substantially the same time as, or prior to, or after an effective amount of one or more of the WNT/β-catenin signal transduction inhibitors of use in the invention; or
(b1) contacting said DCs with an effective amount of one or more of the WNT/β-catenin signal transduction inhibitors of use in the invention, and
(b2) administering the inhibitor-treated DCs to the subject.

In option (b), the method may further comprise a step in which an effective amount of the one or more inhibitors is administered to the subject at the same time as, or substantially the same time as, or prior to, or after the inhibitor treated DCs.

The DCs may be autologous and thus the method may comprise a preceding step of isolating (e.g. harvesting and enriching) DCs or DC precursor cells from the subject. In the latter case the method may comprise a step of inducing said precursor cells to develop into DCs prior to administration. In these embodiments the autologous DCs may be described as being ex vivo DCs.

At the time of administration the DCs may be immature, or mature (or maturing) DCs which have been exposed to cancer antigens. An immature DC is a DC in which there us essentially or substantially no expression of (i.e. low levels of expression of) each of the following: the mature DC marker CD83, the costimulatory molecules CD40, CD80, and CD86, and/or the class II MHC Ag-presenting molecule HLA-DR (Vieira et. al., J. Immunol. 184:4507-4512 (2000)). In other embodiments an immature DC is a DC which is not expressing, or expressing essentially or substantially none of (i.e. low levels of expression of), one or more of the foregoing, especially CD83. In further embodiments an immature DC is a DC which is also not expressing all of TNFα, TGFβ, IL-1, IL-6, IL-10 and IL-18m. In still further embodiments an immature DC is a DC which is also not expressing any of TNFα, TGFβ, IL-1, IL-6, IL-10 and IL-18m. In more detailed embodiments an immature DC will also not express CD14, CD3, CD19, CD16+CD56 and CD66b. A mature DC may be defined in contrast to the definition of an immature DC.

The DCs may be administered systemically or locally, in particularly intratumorally or to the site of the tumour following ablation (e.g. cryoablation) or other such destruction. In embodiments in which the subject receives one or more WNT/β-catenin signal transduction inhibitors of use in the invention, these may be administered in the same way and/or to the same location. The inhibitors can of course be administered via different routes.

In these contexts an effective amount of the WNT/β-catenin signal transduction inhibitor is that amount which is sufficient to combat (e.g. reduce, abrogate, reverse or eliminate) the pro-cancer WNT/β-catenin signalling described above.

Included within the scope of “substantially the same time” is administration of the one or more WNT/β-catenin signal transduction inhibitors immediately or almost immediately before or after the DCs. The term “almost immediately” may be read as including application within one hour of the previous application, preferably within 30 minutes. However the one or more WNT/β-catenin signal transduction inhibitors may be administered at least 1 hour, at least 3 hours, or at least 6 hours or more before the DCs. In these embodiments the DCs can be applied or administered with or without a further application of the one or more WNT/β-catenin signal transduction inhibitors. The one or more WNT/β-catenin signal transduction inhibitors can be applied or administered in a plurality of applications prior to or with the DCs, including as noted above, an application or administration immediately or almost immediately after the DCs. In other embodiments the DCs may conveniently be applied or administered before the one or more WNT/β-catenin signal transduction inhibitors, e.g. at least 1 hour, at least 3 hours, at least 6 hours before the one or more WNT/β-catenin signal transduction inhibitors. In these embodiments the one or more WNT/β-catenin signal transduction inhibitors can be administered with or without a further application of DCs. The DCs can be applied or administered in a plurality of applications prior to, or with, the one or more WNT/β-catenin signal transduction inhibitor, including as noted above, an application or administration immediately or almost immediately after the one or more WNT/β-catenin signal transduction inhibitors.

In preferred embodiments at least one administration of the one or more WNT/β-catenin signal transduction inhibitors is timed to coincide with the priming phase of the DC induced anti-cancer immune response e.g. at least about 1, 2, 3, 4 or 5 days and/or e.g. no more than about 10, 8, 6, 5, 4, 3, 2 or 1 days following the first or each application of DCs.

In these embodiments the one or more of WNT/β-catenin signal transduction inhibitors are administered systemically, e.g. orally, intravenously, subcutaneously, intradermally, or locally, e.g. intratumorally or to the site of the tumour following ablation (e.g. cryoablation) or other such destruction.

The hyperproliferative or neoplastic disease or condition may be any of those recited above, but not necessarily with limitation to those in which WNT/β-catenin signal transduction in a neoplastic cell is a contributing factor. It is however preferred that the hyperproliferative or neoplastic disease or condition be any of those recited above and in which WNT/β-catenin signal transduction in a neoplastic cell is a contributing factor.

In embodiments of the present invention in which axitinib is used, the hyperproliferative or neoplastic disease or condition undergoing treatment or being prevented is preferably not renal cell carcinoma and/or the subject preferably does not have renal cell carcinoma. In other embodiments in which axitinib is used, the hyperproliferative or neoplastic disease or condition undergoing treatment or being prevented is preferably not renal cancer and/or the subject preferably does not have renal cancer. In other embodiments in which axitinib is used, the hyperproliferative or neoplastic disease or condition undergoing treatment or being prevented is preferably not a cancer which has been shown to be or is predicted to be responsive to angiogenesis inhibitors and/or tyrosine kinase inhibitors, in particular inhibitors of vascular endothelial growth factor receptors (1-3), c-KIT, colony stimulating factor-1 (CSF-1) receptor and PDGFR (1 and 2) and/or the subject preferably does not have such a cancer. Such cancers may comprise cancer cells or a tumour which have/has increased levels of, or increased signalling through, one or more of these proteins

In embodiments of the present invention in which pazopanib is used, the hyperproliferative or neoplastic disease or condition is preferably not renal cell carcinoma or soft tissue sarcoma and/or the subject preferably does not have renal cell carcinoma or soft tissue sarcoma. In other embodiments in which pazopanib is used, the hyperproliferative or neoplastic disease or condition is preferably not renal cancer or soft tissue cancer and/or the subject preferably does not have renal cancer or soft tissue cancer. In other embodiments in which pazopanib is used, the hyperproliferative or neoplastic disease or condition is preferably not a cancer which has been shown to be or is predicted to be responsive to angiogenesis inhibitors and/or tyrosine kinase inhibitors, in particular inhibitors of vascular endothelial growth factor receptors (1-3), FGFR (1 and 3) and PDGFR (1 and 2) and/or the subject preferably does not have such a cancer. Such cancers may comprise cancer cells or a tumour which have/has increased levels of, or increased signalling through, one or more of these proteins

In certain embodiments of the present invention in which topotecan is used, the hyperproliferative or neoplastic disease or condition is preferably not small-cell lung cancer, cervical cancer or ovarian cancer and/or the subject preferably does not have small-cell lung cancer, cervical cancer or ovarian cancer. In other embodiments in which topotecan is used, the hyperproliferative or neoplastic disease or condition is preferably not lung cancer and/or the subject preferably does not have lung cancer. In other embodiments in which topotecan is used, the hyperproliferative or neoplastic disease or condition is preferably not a cancer which has been shown to be or is predicted to be responsive to DNA replication inhibitors, in particular inhibitors of topoisomerase I and/or the subject preferably does not have such a cancer.

In certain embodiments of the present invention in which orlistat is used, the hyperproliferative or neoplastic disease or condition is preferably not a cancer which has been shown to be or is predicted to be responsive to inhibitors of fatty acid synthase and/or the subject preferably does not have such a cancer. Such cancers may comprise cancer cells or a tumour which have/has increased levels, or increased activity, of fatty acid synthase.

In further embodiments of the invention the diseases or conditions treated or prevented in accordance with the invention may be diseases or conditions in which WNT/β-catenin signal transduction in an immune cell is a contributing factor. Immune cells are considered to be leukocytes, e.g. the phagocytes (e.g. monocytes, macrophages, neutrophils, DCs, mast cells), the lymphocytes (e.g. natural killer (NK) cells, T cells and B cells), eosinophils and basophils. B cells may for instance be plasma B cells or memory B cells. T cells may for instance be regulatory T cells (T_{reg}), effector T cells (e.g. helper T (T_{H}) cells, cytotoxic T cells (Tₐ, cytotoxic T lymphocytes, CD8+ T cells)), memory T cells, natural killer T cells, mucosal associated invariant T cells and gamma delta T cells. In certain embodiments the immune cell is a DC, a T_{reg}, or a T_{C}, preferably a DC, a T_{reg} cell.

In these embodiments the method of the invention may be considered to be a method for the treatment or prevention of an immune or inflammatory disease or condition in which WNT/β-catenin signal transduction is a contributing factor. The immune or inflammatory disease or condition may be further described as an immune or inflammatory disease or condition which is mediated by, promoted by, caused by, associated with, or which involves or results from WNT/β-catenin signal transduction.

In more specific embodiments said WNT/β-catenin signal transduction is in one or more of the abovementioned immune cells. For instance, the WNT/β-catenin signal transduction may be signalling in effector T cells (e.g. cytotoxic T lymphocytes) and/or T_{Reg} cells which leads to the imprinting of proinflammatory properties on said cells. In other specific instances the WNT/β-catenin signal transduction may be signalling that influences (regulates) tolerogenic signalling in DCs and which promotes the switch from a tolerogenic state to a proinflammatory state.

In certain embodiments there may be increased WNT/β-catenin signal transduction compared to normal (i.e. the levels of WNT/β-catenin signal transduction in an unstimulated immune cell). This may be referred to as overactivity in the WNT/β-catenin signal transduction pathway in the abovementioned immune cells. This may be expressed as proinflammatory WNT/β-catenin signalling and as such the method of the invention may be considered to be a method for the treatment or prevention of an immune or inflammatory disease or condition in which proinflammatory WNT/β-catenin signalling is a contributing factor.

In more specific embodiments the immune or inflammatory disease or condition may be an autoimmune disease (e.g. rheumatoid arthritis, psoriatic arthritis, psoriasis, diabetes mellitus type 1, celiac disease, Crohn's disease, microscopic colitis, ulcerative colitis, multiple sclerosis, myocarditis, autoimmune hepatitis, lupus, alopecia areata, autoimmune progesterone dermatitis, autoimmune urticarial, autoimmune polyendocrine syndrome 1, 2 and 3, autoimmune pancreatitis, autoimmune thyroiditis, Sjogren's syndrome, antiphospholipid syndrome, autoimmune hemolytic anemia, autoimmune lymphoproliferative syndrome, mixed connective tissue disease, relapsing polychondritis, rheumatic fever, undifferentiated connective tissue disease, dermatomyositis, polymyositis, Guillain-Barré syndrome, multiple sclerosis, vasculitis), inflammatory bowel disease, colitis, atherosclerosis, neurodegeneration, neuroinflammation, allograft organ transplant rejection, allergy, a chronic microbial (e.g. bacterial, fungal, virus and/or protozoan) infection or a chronic wound, preferably rheumatoid arthritis, psoriatic arthritis, psoriasis, diabetes mellitus type 1, celiac disease, colitis (e.g. Crohn's disease, ulcerative colitis, inflammatory bowel disease) multiple sclerosis, atherosclerosis, neurodegeneration, neuroinflammation, allograft organ transplant rejection, allergy, a chronic microbial infection or a chronic wound.

In certain embodiments the immune or inflammatory disease or condition is not a hyperproliferative or neoplastic disease or condition, e.g. a cancer, in particular the cancers mentioned above (e.g. colorectal cancer and prostate cancer), whether dependent on WNT/β-catenin signal transduction or otherwise. In other embodiments the immune or inflammatory disease or condition is not mediated by, promoted by, caused by, associated with, nor which involves or results from a hyperproliferative or neoplastic disease or condition, e.g. a cancer, in particular the cancers mentioned above (e.g. colorectal cancer and prostate cancer), whether dependent on WNT/β-catenin signal transduction or otherwise. In still further embodiments the subject does not have a hyperproliferative or neoplastic disease or condition, e.g. a cancer, in particular the cancers mentioned above (e.g. colorectal cancer and prostate cancer), whether dependent on WNT/β-catenin signal transduction or otherwise.

WNT/β-catenin signal transduction has also been recognised recently as a contributing factor in certain disorders and dysfunctions in the metabolism of carbohydrates. In the context of diabetes mellitus type 2, gain of function mutations in certain β-catenin activated transcription factors have shown to be a risk factor for diabetes mellitus type 2 and thus insulin resistance, metabolic syndrome and obesity.

Thus, in further embodiments of the invention the disease or condition treated or prevented in accordance with the invention may be a disorder or dysfunction in the metabolism of carbohydrates by a subject, e.g. diabetes mellitus type 2, insulin resistance, metabolic syndrome, obesity and diabetic retinopathy, nephropathy and neuropathy. In certain embodiments, in particular those involving orlistat, the disease or condition treated or prevented in accordance with the invention is not obesity.

WNT/β-catenin signal transduction has also been recognised recently as playing a role in the process of wound healing, in particular cutaneous wound healing. It has been found that the migration and proliferation of keratinocytes and fibroblasts may be regulated by WNT/β-catenin signal transduction and that the other key signalling pathways, e.g. TNF-β mediated pathways, may be influenced by WNT/β-catenin signal transduction. Moreover the process of wound healing has an inflammatory component and as such the role WNT/β-catenin signal transduction plays in wound healing may also be effected through regulation of immune cells in the wound and surrounding tissues.

In accordance with the invention, a wound may be considered to be a breach in, or denudement of, a tissue. Wounds may be formed by trauma. Wounds may also be surgical. Wounds may also be caused by a spontaneously forming lesion such as a skin ulcer (e.g. a venous, diabetic or pressure ulcer), an anal fissure or a mouth ulcer. The term “trauma” refers broadly to cellular attack by foreign bodies and/or physical injury of cells. Included among foreign bodies are microorganisms, particulate matter, chemical agents, and the like. Included among physical injuries are mechanical injuries; thermal injuries (burns/scalds), such as those resulting from excessive heat or cold; electrical injuries, such as those caused by contact with sources of electrical potential; and radiation damage.

Wounds are typically defined as either acute or chronic. Acute wounds are wounds that proceed orderly through the three recognised stages of the healing process (i.e. the inflammatory stage, the proliferative stage and the remodelling phase) without a protracted timecourse. Chronic wounds, however, are those wounds that do not complete the ordered sequence of biochemical events of the healing process because the wound has stalled in one of the healing stages. Commonly, chronic wounds are stalled in the inflammatory phase. In accordance with a particular aspect of the present invention, a chronic wound may be considered to be a wound that has not healed in the expected amount of time, e.g. at least 5, 10, 15, 20 or 30 days longer than expected. This may be taken as a wound that has not healed at least 30 days, at least 40 days, particularly at least 50 days, more particularly at least 60 days, most particularly at least 70 days after formation.

Through the inhibition of WNT/β-catenin signal transduction in the context of wound healing, e.g. more specifically through down-regulating the migration and overproliferation of keratinocytes and fibroblasts or the inflammatory activities of immune cells in the wound and surrounding tissues, the WNT/β-catenin signal transduction inhibitors of use in the invention may be effective in promoting the healing of chronic wounds.

Thus, in further embodiments of the invention the disease or condition treated in accordance with the invention may be a chronic wound.

Expressed differently, the method of the invention may be a method to promote the healing of a chronic wound, wherein one or more of the WNT/β-catenin signal transduction inhibitors of use in the invention are administered to a subject with a chronic wound, more specifically wherein one or more of the WNT/β-catenin signal transduction inhibitors of use in the invention is applied to said chronic wound, in an amount sufficient to promote the healing of the chronic wound.

By promotion of healing it is meant that the treatment accelerates the healing process of the chronic wound in question (i.e. the progression of the wound through the three recognised stages of the healing process). The acceleration of the healing process may manifest as an increase in the rate of progression through one, two or all of the healing stages (i.e. the inflammatory stage, the proliferative stage and/or the remodelling phase). If the chronic wound is stalled in one of the healing stages the acceleration might manifest as the restarting of the linear, sequential healing process after the stall. In other words, the treatment shifts the chronic wound from a non-healing state to a state where the wound begins to progress through the healing stages. That progression after the restart may be at a normal rate or even a slower rate compared with the rate a normal acute wound would heal. Promotion of wound healing may also be considered to amount to the prevention of a further or continued deceleration the healing process of the chronic wound in question. A deceleration of the healing process may manifest as a decrease in the rate of progression through one, two or all of the healing stages. If the chronic wound is restarting on the linear, sequential healing process after a stall deceleration might manifest as a return to being stalled in one of the healing stages. In other words, the treatment prevents a chronic wound that is beginning to heal from shifting to a non-healing state.

The chronic wound may be found in or on a subject. The term “in a subject” is used broadly herein to include sites or locations inside a subject or on a subject, e.g. an external body surface, and may include in particular a wound containing an implantable a medical device.

Thus, the chronic wound may therefore be found, for instance, in or on the skin or in or on any susceptible surface in the oral cavity (e.g. gingiva, gingival crevice, periodontal pocket), the reproductive tract (e.g. cervix, uterus, fallopian tubes), the peritoneum, the gastrointestinal tract, the ear, the eye, the prostate, the urinary tract, the vascular system, the respiratory tract, the heart, the kidney, the liver, the pancreas, the nervous system or the brain. Preferably the chronic wound is a skin (cutaneous) wound, in other words a dermal or dermatological wound, which includes wounds to any depth of the epidermis and/or dermis and the underlying tissue.

WNT/β-catenin signal transduction has also been recognised as a contributing factor in high bone mass disorders and sclerosteosis where it positively regulates osteoblast proliferation.

Thus, in further embodiments of the invention the disease or condition treated or prevented in accordance with the invention may be a high bone mass disorder or sclerosteosis.

The invention further provides each of axitinib, pazopanib, orlistat and/or topotecan, or any combination thereof, for use in the therapeutic methods described herein. All details described in connection with those methods apply mutatis mutandis to this aspect of the invention.

The invention further provides the use of each of axitinib, pazopanib, orlistat and/or topotecan, or any combination thereof, in the manufacture of a medicament for use in the therapeutic methods described herein. All details described in connection with those methods apply mutatis mutandis to this aspect of the invention.

The skilled person is able to identify diseases or conditions in which WNT/β-catenin signal transduction is a contributing factor, specifically for example the more particularly defined diseases or conditions recited herein, without undue burden. For instance, as described in the Examples, molecular probes are available which emit a signal in the presence of WNT signalling and may be detected by, inter alia, microscopy and flow cytometry. Selective inhibitors are available as described in Takebe et al. supra, the contents of which are incorporated by reference, and activators (e.g. 6BIO) of the WNT/β-catenin signal transduction pathway are also available and may be used to determine the reliance of a disease phenotype or phenotype of a particular cell (e.g. a neoplastic cell or an immune cell) on WNT/β-catenin signal transduction. The intracellular (e.g. nuclear vs cytoplasmic) localisation of β-catenin in the relevant cells or tissues can also be monitored to reveal levels of WNT/β-catenin signal transduction, e.g. by immunocytochemistry and suitable microscopy techniques. Likewise, real time analysis of the activation of β-catenin activated genes, e.g. by qPCR, DNA microarrays or RNA-sequencing may also be used to reveal excessive or inappropriate WNT/β-catenin signal transduction in a disease state. The methods of determining upregulation of WNT/β-catenin signal transduction in cells described in US 2014/0113006 may also be used.

Moreover, in the particular context of the treatment or prevention of neoplastic diseases and conditions, genetic analysis of the genome of a target neoplastic cell may be performed to reveal the presence or absence of mutations in the components of the WNT/β-catenin signal transduction pathway, e.g. those described herein.

In certain embodiments the methods of the invention comprise a preceding step in which the subject is determined, e.g. diagnosed, as having or being at risk of a disease or condition in which WNT/β-catenin signal transduction is a contributing factor, specifically for example the more particularly defined disease or conditions recited herein, as appropriate. By way of example, the method of the invention for the treatment or prevention of a hyperproliferative or neoplastic disease or condition in which WNT/β-catenin signal transduction in a neoplastic cell is a contributing factor may comprise a preceding step in which the subject is determined, e.g. diagnosed, as having or being at risk of a hyperproliferative or neoplastic disease or condition in which WNT/β-catenin signal transduction in a neoplastic cell is a contributing factor. This determination may be achieved through the use of the above described approaches.

In other embodiments the method comprises a (further) preceding step in which the subject is determined, e.g. diagnosed, as not having or not being at risk of the disclaimed cancers recited above, in particular those cancers determined as being responsive to certain classes of anticancer agents or having certain markers, as defined above).

In other embodiments the methods of the invention may further comprise a following step in which the subject's clinical indictors of the disease or condition in which WNT/β-catenin signal transduction is a contributing factor, specifically for example the more particularly defined diseases or conditions recited herein, are assessed and preferably compared to a corresponding assessment made prior to, or earlier in, said treatment in order to determine any changes therein. However, also assessed may be parameters relating to the effect of the WNT/β-catenin signal transduction inhibitors of use in accordance with the invention on the WNT/β-catenin signal transduction which contributes to the subject's disease or condition being treated or prevented in accordance with the invention.

The subject may be any human or non-human animal subject, but more particularly may be a human or non-human vertebrate, e.g. a non-human animal selected from mammals, birds, amphibians, fish and reptiles. The non-human animal may be a livestock or a domestic animal or an animal of commercial value, including laboratory animals or an animal in a zoo or game park. Representative non-human animals therefore include dogs, cats, rabbits, mice, guinea pigs, hamsters, horses, pigs, sheep, goats, cows, chickens, turkeys, guinea fowl, ducks, geese, parrots, budgerigars, pigeons, salmon, trout, tilapia, catfish, bream, barramundi, grouper, mullet, amberjack, croaker, rohu, goby, cod, haddock, sea bass and carp. Veterinary uses of the invention are thus covered. The subject may be viewed as a patient. Preferably the subject is a human.

In certain embodiments, e.g. when axitinib, pazopanib and/or topotecan is selected as the WNT/β-catenin signal transduction inhibitor(s) the subject is not a subject with a hyperproliferative or neoplastic disease or condition, e.g. a cancer.

In certain embodiments, e.g. when orlistat is selected as the WNT/β-catenin signal transduction inhibitor, the subject is not an obese (BMI of greater than or equal to 30) or overweight subject (BMI of greater than or equal to 25).

“Treatment” when used in relation to the treatment of a disease or medical condition (e.g. a wound or a cancer) or disorder or dysfunction in a subject in accordance with the invention is used broadly herein to include any therapeutic effect, i.e. any beneficial effect on, or in relation to, the disease or medical condition or disorder or dysfunction. For brevity in the following the term “condition” specifically encompasses “disease or medical condition or disorder or dysfunction”. Thus, not only included is eradication or elimination of the condition, or cure of the subject of the condition, but also an improvement in the condition of the subject or a halt to a deterioration in the condition. Thus included for example, is an improvement in any symptom or sign of the condition, or in any clinically accepted indicator of the condition (for example a decrease in tumour size (volume, area and/or cell number), a decrease in tumour invasion or a reduction in general discomfort or pain in the surrounding tissue). Treatment thus includes both curative and palliative therapy, e.g. of a pre-existing or diagnosed condition, i.e. a reactionary treatment.

“Prevention” as used herein refers to any prophylactic or preventative effect. It thus includes delaying, limiting, reducing or preventing the condition or the onset of the condition, or one or more symptoms or indications thereof (e.g. an increase in the size of a tumour or the development of secondary tumours), for example relative to the condition or symptom or indication prior to the prophylactic treatment. Prophylaxis thus explicitly includes both absolute prevention of occurrence or development of the condition, or symptom or indication thereof, and any delay in the onset or development of the condition or symptom or indication, or reduction or limitation on the development or progression of the condition or symptom or indication.

Specifically, the WNT/β-catenin signal transduction inhibitors of use in the invention can be taken as a prophylactic treatment, for example to prevent, or at least minimise the risk of, the diseases, conditions, disorders or dysfunctions described herein.

In the therapeutic methods of the invention the WNT/β-catenin signal transduction inhibitors of use in the invention will be administered (applied) to the subject in “pharmaceutically effective” or “physiologically effective” (which terms may be used interchangeably with each other and “therapeutically effective”) amounts. A “pharmaceutically/physiologically effective” amount of the WNT/β-catenin signal transduction inhibitors of use in the invention is that amount of inhibitor which provides measurable treatment or prevention of the target diseases, conditions, disorders or dysfunctions described herein. This may also be expressed as a therapeutically or prophylatically effective reduction in the WNT/β-catenin signal transduction which contributes to the target diseases, conditions, disorders or dysfunctions described herein, e.g. in the specific cells described above. This may, in some embodiments, be a reduction in normal levels of WNT/β-catenin signal transduction to below normal levels, or a reduction in increased or elevated levels of WNT/β-catenin signal transduction compared to normal, e.g. to normal levels or below normal levels. The skilled person would be able to appreciate what levels of WNT/β-catenin signal transduction would be considered normal from time to time and thus would appreciate levels which are increase/elevated or below normal.

A “pharmaceutically effective” or “physiologically effective” amount can be determined with reference to standard practices for deciding dosage amounts and the skilled person will be able to detect evidence of successful treatment from his experience and with the aid of routine tests available to him that are designed to monitor the targeted condition and levels of WNT/β-catenin signal transduction.

Suitable doses of the WNT/β-catenin signal transduction inhibitors of use in the invention which may achieve the pharmaceutically/physiologically effective amounts will therefore vary from subject to subject and can be determined by the physician or veterinary practitioner in accordance with the weight, age and sex of the subject, the severity of the condition and the mode of administration. Moreover, each WNT/β-catenin signal transduction inhibitor of use in the invention is an approved drug and as such comprehensive safety and efficacy data are available for each inhibitor to guide the skilled person in this regard.

It will be clear from the foregoing that by administering or applying a WNT/β-catenin signal transduction inhibitor of use in the invention as herein defined to a subject and achieving the requisite therapeutic effect, the method comprises a step wherein cells, the WNT/β-catenin signal transduction within which is a contributing factor to the disease or condition being targeted for treatment, are contacted with an amount of inhibitor sufficient to reduce the WNT/β-catenin signal transduction in said cells. These cells may be those specifically recited herein, e.g. neoplastic cells and/or immune cells. This contacting step may be achieved by any convenient and technically appropriate means of drug administration, e.g. those described in detail below, in particular by oral administration, systemic injection or direct injection into the target treatment site.

In one embodiment of the invention the WNT/β-catenin signal transduction inhibitors of use in the invention as herein defined may be used in the methods or uses of the invention in conjunction or combination with (together with) a further pharmaceutical for the treatment of said disease or condition in which WNT/β-catenin signal transduction is a contributing factor

The further pharmaceutical (i.e. further therapeutically active agent) may be a cytotoxic chemotherapy agent, an angiogenesis inhibitor, an anti-cancer monoclonal antibody, a radioimmunotherapeutic, a cancer treatment vaccine, an immunostimulatory agent, an immunosuppressant, a corticosteroid, a non-steroidal anti-inflammatory drug (NSAID), an antibiotic, an antifungal, an antiviral, an oral antidiabetic drug or an injectable antidiabetic drug.

The further pharmaceutical does not include any or all of axitinib, pazopanib, orlistat, topotecan, pharmaceutically effective substitution derivatives thereof wherein one or more hydrogen groups are substituted with SR¹ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, or pharmaceutically acceptable salts, or solvates or hydrates thereof, diastereoisomers, tautomers, enantiomers, and prodrugs and active metabolites thereof.

Representative examples of suitable cytotoxic chemotherapy agents include, but are not limited to, bleomycin, capecitabine, carboplatin, cisplatin, cyclophosphamide, dacarbazine, docetaxel, doxorubicin, pegylated liposomal doxorubicin, epirubicin, eribulin, etoposide, fluorouracil, gemcitabine, ixabepilone, methotrexate, mechlorethamine, oxaliplatin, paclitaxel, procarbazine, prednisolone, protein-bound paclitaxel, vinorelbine, vinblastine and vincristine.

Representative examples of suitable angiogenesis inhibitors include, but are not limited to, bevacizumab, everolimus, lenalidomide, ramucirumab sorafenib, sunitinib and thalidomide.

Representative examples of suitable anti-cancer monoclonal antibody include, but are not limited to, alemtuzumab, bevacizumab, cetuximab, ofatumumab, panitumumab, rituximab, and trastuzumab.

Representative examples of suitable radioimmunotherapeutics include, but are not limited to, ibritumomab and tositumomab.

Representative examples of suitable cancer treatment vaccines include, but are not limited to, sipuleucel-T.

Representative examples of immunostimulatory agents include, but are not limited to, cytokines e.g. TNF, IFNα, IL-1, IL-2, IL-6 and IL-8.

Representative examples of suitable immunosuppressants include, but are not limited to, cyclosporine, rapamycin, tacrolimus, dactinomycin, mitomycin c, bleomycin, mithramycin, azathioprine, hydrocortisone, cortisone, prednisone, prednisolone, methylprednisolone, bexamethasone, betamethasone, triamcinolone, beclomethasone, fludrocortisone acetate, deoxycorticosterone acetate and aldosterone.

Representative examples of suitable corticosteroids include, but are not limited to, prednisone, flunisolide, triamcinolone, fluticasone, budesonide, mometasone, beclomethasone, amcinonide, budesonide, desonide, fluocinonide, fluocinolone, halcinonide, hydrocortisone, cortisone, tixocortol, prednisolone, methylprednisolone, prednisone, betamethasone, dexamethasone, fluocortolone, aclometasone, prednicarbate, clobetasone, clobetasol, and fluprednidene.

Representative examples of suitable NSAIDs include, but are not limited to, the salicylates (e.g. aspirin (acetylsalicylic acid), choline magnesium trisalicylate, diflunisal, salsalate, the propionic acid derivatives (e.g. ibuprofen, dexibuprofen, dexketoprofen, fenoprofen, flurbiprofen, ketoprofen, loxoprofen, naproxen, oxaprozin), the acetic acid derivatives (e.g. aceclofenac, diclofenac, etodolac., indomethacin, ketorolac, nabumetone, tolmetin, sulindac), the enolic acid derivatives (e.g. droxicam, isoxicam, lornoxicam, meloxicam, piroxicam, tenoxicam), the anthranilic acid derivatives (e.g. flufenamic acid, meclofenamic acid, mefenamic acid, tolfenamic acid) and the selective COX-2 inhibitors (Coxibs; e.g. celecoxib, etoricoxib, lumiracoxib, parecoxib, rofecoxib, valdecoxib). The propionic acid derivatives (e.g. ibuprofen, dexibuprofen, dexketoprofen, fenoprofen, flurbiprofen, ketoprofen, loxoprofen, naproxen, oxaprozin) are preferred, ibuprofen being most preferred.

The antibiotic may be selected from the aminoglycosides (e.g. amikacin, gentamicin, kanamycin, neomycin, netilmicin, streptomycin, tobramycin); the β-lactams (e.g. the carbecephems (e.g. loracarbef); the 1st generation cephalosporins (e.g. cefadroxil, cefazolin, cephalexin); 2nd generation cephalosporins (e.g. cefaclor, cefamandole, cephalexin, cefoxitin, cefprozil, cefuroxime); 3rd generation cephalosporins (e.g. cefixime, cefdinir, cefditoren, cefoperazone, cefotaxime, cefpodoxime, ceftazidime, ceftibuten, ceftizoxime, ceftriaxone); 4th generation cephalosporins (e.g. cefepime); the monobactams (e.g. aztreonam); the macrolides (e.g. azithromycin, clarithromycin, dirithromycin, erythromycin, troleandomycin); the monobactams (e.g. aztreonam); the penicillins (e.g. amoxicillin, ampicillin, carbenicillin, cloxacillin, dicloxacillin, nafcillin, oxacillin, penicillin G, penicillin V, piperacillin, ticarcillin); the polypeptide antibiotics (e.g. bacitracin, colistin, polymyxin B); the quinolones (e.g. ciprofloxacin, enoxacin, gatifloxacin, levofloxacin, lomefloxacin, moxifloxacin, norfloxacin, ofloxacin, trovafloxacin); the sulfonamides (e.g. mafenide, sulfacetamide, sulfamethizole, sulfasalazine, sulfisoxazole, trimethoprim-sulfamethoxazole); the tetracyclines (e.g. demeclocycline, doxycycline, minocycline, oxytetracycline, tetracycline); the glycylcyclines (e.g. tigecycline); the carbapenems (e.g. imipenem, meropenem, ertapenem, doripenem, panipenem/betamipron, biapenem, PZ-601); other antibiotics include chloramphenicol; clindamycin, ethambutol; fosfomycin; isoniazid; linezolid; metronidazole; nitrofurantoin; pyrazinamide; quinupristin/dalfopristin; rifampin; spectinomycin; and vancomycin.

Representative examples of suitable antibiotics include, but are not limited to, amikacin, gentamicin, kanamycin, neomycin, netilmicin, streptomycin, tobramycin, cefixime, cefdinir, cefditoren, cefoperazone, cefotaxime, cefpodoxime, ceftazidime, ceftibuten, ceftizoxime, ceftriaxone, cefepime, aztreonam, amoxicillin, ampicillin, carbenicillin, cloxacillin, dicloxacillin, nafcillin, oxacillin, penicillin G, penicillin V, piperacillin, ticarcillin, ciprofloxacin, enoxacin, gatifloxacin, levofloxacin, lomefloxacin, moxifloxacin, norfloxacin, ofloxacin, trovafloxacin, azithromycin, clarithromycin, dirithromycin, erythromycin, roxithromycin, telithromycin, CarbomycinA, josamycin, kitasamycin, midecamicine, oleandomycin, spiramycin, troleandromycin, tylosin, imipenem, meropenem, ertapenem, doripenem, panipenem/betamipron, biapenem, PZ-601, bacitracin, colistin, polymyxin B, demeclocycline, doxycycline, minocycline, oxytetracycline and tetracycline.

Representative examples of suitable antifungals include, but are not limited to the polyenes (e.g. natamycin, rimocidin, filipin, nystatin, amphotericin B, candicin; the imidazoles (e.g. miconazole, ketoconazole, clotrimazole, econazole, bifonazole, butoconazole, fenticonazole, isoconazole, oxiconazole, sertaconazole, sulconazole, tioconazole); the triazoles (e.g. fluconazole, itraconazole, isavuconazole, ravuconazole, posaconazole, voriconazole, terconazole); the allylamines (e.g. terbinafine, amorolfine, naftifine, butenafine); and the echinocandins (e.g. anidulafungin, caspofungin, micafungin).

Representative examples of suitable antivirals include, but are not limited to abacavir, acyclovir, adefovir, amantadine, amprenavir, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, nelfinavir, nevirapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

Representative examples of suitable oral antidiabetic drugs include, but are not limited to, the sulfonylureas (e.g. carbutamide, acetohexamide, chlorpropamide, tolbutamide, glipizide, gliclazide, glibenclamide, glibornuride, gliquidone, glisoxepide, glyclopyramide, glimepiride), the biguanides (e.g. metformin, phenformin, buformin, proguanil), the thiazolidinediones (e.g. rosiglitazone, pioglitazone, troglitazone), the alpha-glucosidase inhibitors (e.g. acarbose, miglitol, voglibose), the meglitinides (e.g. nateglinide, repaglinide, mitiglinide), and the glycosurics (e.g. dapagliflozin, ganagliflozin, ipragliflozin, tofogliflozin, empagliflozin, sergliflozin etabonate, remogliflozin etabonate).

Representative examples of suitable injectable antidiabetic drugs include, but are not limited to, insulin and its analoges (e.g. insulin lispro, insulin aspart, insulin glulisine, insulin zinc, isophane insulin, insulin glargine, insulin detemir) and the incretin mimetics (e.g. the glucagon-like peptide (GLP) agonists, e.g. exenatide, liraglutide, and taspoglutide; and the dipeptidyl peptidase-4 (DPP-4) inhibitors, e.g. vildagliptin, sitagliptin, saxagliptin, linagliptin, allogliptin and septagliptin).

The further pharmaceutical for the treatment of said disease or condition in which WNT/β-catenin signal transduction is a contributing factor may conveniently be applied before, simultaneously with, or following the WNT/β-catenin signal transduction inhibitors of use in the invention as herein defined. Conveniently the further pharmaceutical is applied at substantially the same time as the inhibitor or afterwards. In other embodiments the further pharmaceutical may conveniently be applied or administered before the inhibitor. The further pharmaceutical can also be given (e.g. administered or delivered) repeatedly at time points appropriate for the agent used. The skilled person is able to devise a suitable dosage regimen. In long term treatments the inhibitor can also be used repeatedly. The inhibitor can be applied as frequently as the further pharmaceutical, or more or less frequently. The frequency required may depend on the overall nature of the disease or condition.

The WNT/β-catenin signal transduction inhibitors of use in the invention as herein defined and the further pharmaceutical (or further therapeutically active agent), may for example be administered together, in a single pharmaceutical formulation or composition, or separately (i.e. separate, sequential or simultaneous administration). Thus, the WNT/β-catenin signal transduction inhibitors of use in the invention as herein defined and the further pharmaceutical may be combined, e.g. in a pharmaceutical kit or as a combined (“combination”) product.

The invention therefore also provides products (e.g. a pharmaceutical kit or a combined (“combination”) product) or compositions (e.g. a pharmaceutical composition) wherein the product or composition comprises a WNT/β-catenin signal transduction inhibitor of use in the invention as herein defined and a further pharmaceutical (or further therapeutically active agent) for the treatment or prevention of a disease or condition in which WNT/β-catenin signal transduction is a contributing factor, e.g. those described above. Combinations comprising a WNT/β-catenin signal transduction inhibitor of use in the invention as herein defined as herein defined and a cytotoxic chemotherapy agent, an angiogenesis inhibitor, an anti-cancer monoclonal antibody, a radioimmunotherapeutic, a cancer treatment vaccine, an immunostimulatory agent, an immunosuppressant, a corticosteroid, a non-steroidal anti-inflammatory drug (NSAID) or an antibiotic are preferred. Such pharmaceutical products and pharmaceutical compositions are preferably adapted for use in the therapeutic methods of the invention.

The use of the WNT/β-catenin signal transduction inhibitors of use in the invention as herein defined to manufacture such pharmaceutical products and pharmaceutical compositions for use in the therapeutic methods of the invention is also contemplated.

The WNT/β-catenin signal transduction inhibitors of use in the invention as herein defined may be administered to the subject in any convenient form or by any convenient means in order to achieve the requisite inhibition of WNT/β-catenin signal transduction in the target treatment area, e.g. in the target cells discussed above. Such convenient means may include topical, enteral (e.g. oral, buccal, sublingual, rectal), parenteral (e.g. intravenous, intra-arterial, intraosseous, intra-muscular, intracerebral, intrathecal, subcutaneous, intradermal, intrapancreatic, intratumoral or into the remnants of a tumour and/or surrounding tissue following ablation or other such destruction) or inhalation (including nasal inhalation) means of administration.

Preferably the WNT/β-catenin signal transduction inhibitors of use in the invention as herein defined will be administered by enteral routes (in particular oral) or by parenteral routes. Topical administration to exposed treatment areas may also be convenient.

The skilled man will be able to formulate the WNT/β-catenin signal transduction inhibitors of use in the invention as herein defined into pharmaceutical compositions that are adapted for these routes of administration according to any of the conventional methods known in the art and widely described in the literature. Notably the inhibitors of the invention are all approved for oral administration. Compositions for use in the various parenteral administration routes may, at their simplest, be solutions of the inhibitors in sterile water.

The present invention therefore also provides a pharmaceutical composition for use in any of the above-mentioned methods or uses comprising an WNT/β-catenin signal transduction inhibitor of use in the invention as herein defined, together with at least one pharmaceutically acceptable carrier, diluent or excipient, preferably in an amount sufficient to achieve the requisite inhibition of WNT/β-catenin signal transduction in the target treatment area, e.g. in the target cells discussed above. This composition may also comprise other therapeutic agents as described above.

More specifically, the WNT/β-catenin signal transduction inhibitors of use in the invention as herein defined may be incorporated, optionally together with other active agents, with one or more conventional carriers, diluents and/or excipients, to produce conventional galenic preparations such as tablets, pills, powders (e.g. inhalable powders, including dry inhalable powders), lozenges, sachets, cachets, elixirs, suspensions, emulsions, solutions, syrups, aerosols (as a solid or in a liquid medium), sprays (e.g. nasal sprays), compositions for use in nebulisers, ointments, creams, salves, soft and hard gelatine capsules, suppositories, pessaries, sterile injectable solutions, sterile packaged powders, and the like. Enteric coated solid or liquid compositions and sterile injectable compositions are of particular note.

Examples of suitable carriers, excipients, and diluents are lactose, dextrose, sucrose, sorbitol, mannitol, starches, gum acacia, calcium phosphate, inert alginate polymers, tragacanth, gelatine, calcium silicate, microcrystalline cellulose, polyvinylpyrrolidone, cellulose, water syrup, water, water/ethanol, water/glycol, water/polyethylene, hypertonic salt water, glycol, propylene glycol, methyl cellulose, methylhydroxybenzoates, propyl hydroxybenzoates, talc, magnesium stearate, mineral oil or fatty substances such as hard fat or suitable mixtures thereof. Excipients and diluents of note are mannitol and hypertonic salt water (saline).

The compositions may additionally include lubricating agents, wetting agents, emulsifying agents, suspending agents, preserving agents, sweetening agents, stabilising agents, e.g. buffers and antioxidants, flavouring agents, and the like. Additional therapeutically active agents may be included in the pharmaceutical compositions, as discussed above in relation to combination therapies.

Parenterally administrable forms, e.g. solutions suitable for delivery via intravenous, intra-arterial, intraosseous, intra-muscular, intracerebral, intrathecal, subcutaneous, intradermal, intrapancreatic, intratumoral routes or into the remnants of a tumour and/or surrounding tissue following ablation or other such destruction, e.g. by injection or infusion, should be sterile and free from physiologically unacceptable agents, and should have low osmolarity to minimize irritation or other adverse effects upon administration. Thus such solutions should preferably be isotonic or slightly hypertonic, e.g. hypertonic salt water (saline). Suitable vehicles include aqueous vehicles customarily used for administering parenteral solutions such as sterile water for injection, Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975)), which is explicitly incorporated by reference herein in its entirety. The solutions can contain preservatives, antimicrobial agents, buffers and antioxidants conventionally used for parenteral solutions, excipients and other additives which are compatible with the biopolymers and which will not interfere with the manufacture, storage or use of products.

Simple sterile solutions of WNT/β-catenin signal transduction inhibitors of use in the invention as herein defined or simple sterile liquid compositions comprising WNT/β-catenin signal transduction inhibitors of use in the invention as herein defined may be especially convenient for use during surgical procedures and for delivery to the lungs, e.g. by nebuliser, or to the paranasal sinuses, e.g. by a nasal spray device.

Solid or liquid formulations of the WNT/β-catenin signal transduction inhibitors of use in the invention as herein defined may be provided with an enteric coating that prevents degradation in the stomach and/or other parts of the upper GI tract but permits degradation in the lower GI tract, e.g. the small intestine. Such coatings are routinely prepared from polymers including fatty acids, waxes, shellac, plastics, and plant fibres. Specific examples thereof include but are not limited to methyl acrylate-methacrylic acid copolymers, methyl methacrylate-methacrylic acid copolymers, cellulose acetate succinate, hydroxypropyl methylcellulose phthalate, hydroxypropyl methylcellulose acetate succinate (hypromellose acetate succinate), polyvinyl acetate phthalate (PVAP), cellulose acetate trimellitate, and sodium alginate polymer.

For topical administration the WNT/β-catenin signal transduction inhibitors of use in the invention as herein defined can be incorporated into creams, ointments, gels, salves, transdermal patches and the like. Further topical systems that are envisaged to be suitable are in situ drug delivery systems, for example gels where solid, semi-solid, amorphous or liquid crystalline gel matrices are formed in situ and which may comprise the inhibitor. Such matrices can conveniently be designed to control the release of the inhibitor from the matrix, e.g. release can be delayed and/or sustained over a chosen period of time. Such systems may form gels only upon contact with biological tissues or fluids, e.g. mucosal surfaces. Typically the gels are bioadhesive and/or mucoadhesive. Delivery to any body site that can retain or be adapted to retain the pre-gel composition can be targeted by such a delivery technique, e.g. a tumour or the remnants of a tumour and/or surrounding tissue following ablation or other such destruction. Such systems are described in WO 2005/023176), which is explicitly incorporated by reference herein in its entirety.

The one or more WNT/β-catenin signal transduction inhibitors of use in the invention can also be incorporated into wound dressings e.g. woven and non-woven dry fibrous (e.g. fabric) dressings, film-based dressings, gel-based dressings or dressings which are a combination of these dressings types. The inhibitors may be applied to the dressing prior to or during application to a wound or may be incorporated during manufacture.

The compositions and products of use in the invention will typically comprise 1% to 99%, 2% to 98%, 5% to 95%, 10% to 90%, 15% to 85% or 25% to 75% w/w or w/v (as appropriate) of the WNT/β-catenin signal transduction inhibitors of use in the invention as herein defined, allowance being made for other ingredients, e.g. further therapeutic agents.

The precise content of the WNT/β-catenin signal transduction inhibitors of use in the invention as herein defined in the compositions and products of the invention can vary depending on the dosage required and the dosage regime being followed but will be in an amount sufficient to achieve the requisite inhibition of WNT/β-catenin signal transduction in the target treatment area, e.g. in the target cells discussed above, taking account of variables such as the physical size of the subject to be treated, the nature of the subject's particular ailments, and the location and identity of the target treatment area. The skilled man would know that the amounts of inhibitor can be reduced if a multiple dosing regime is followed or increased to minimise the number of administrations or applications.

A representative topical formulation, e.g. a cream, ointment or salve, which may be used to administer an WNT/β-catenin signal transduction inhibitor of use in the invention as herein defined to the skin might contain 1 to 25%, 1 to 20%, 1 to 15%, 1 to 10%, 1 to 9%, 1 to 8%, 1 to 7%, 1 to 6%, 1 to 5%, 1 to 2%, 2 to 25%, 2 to 20%, 2 to 15%, 2 to 10%, 2 to 9%, 2 to 8%, 2 to 7%, 2 to 6%, 2 to 5%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v of the inhibitor, the remainder being comprised of pharmaceutically acceptable excipients, and/or other active agents if being used.

A representative tablet to be used to administer a WNT/β-catenin signal transduction inhibitor of use in the invention as herein defined systemically may contain 1 to 25%, 1 to 20%, 1 to 15%, 1 to 10%, 1 to 9%, 1 to 8%, 1 to 7%, 1 to 6%, 1 to 5%, 1 to 2%, 2 to 25%, 2 to 20%, 2 to 15%, 2 to 10%, 2 to 9%, 2 to 8%, 2 to 7%, 2 to 6%, 2 to 5%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v or w/w of the inhibitor, the remainder being comprised of pharmaceutically acceptable excipients and/or other active agents if being used.

An enteric coated tablet may also be effective in administering a WNT/β-catenin signal transduction inhibitor of use in the invention as herein defined to the lower GI tract. A representative enteric coated tablet may contain up to contain 1 to 25%, 1 to 20%, 1 to 15%, 1 to 10%, 1 to 9%, 1 to 8%, 1 to 7%, 1 to 6%, 1 to 5%, 1 to 2%, 2 to 25%, 2 to 20%, 2 to 15%, 2 to 10%, 2 to 9%, 2 to 8%, 2 to 7%, 2 to 6%, 2 to 5%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v or w/w of the inhibitor, the remainder being comprised of pharmaceutically acceptable excipients, including the enteric coating (e.g. polymers including fatty acids, waxes, shellac, plastics, and plant fibres) and/or other active agents if being used.

A representative aqueous solution for parenteral delivery, e.g. those routes recited above, will be sterile and may contain 1 to 25%, 1 to 20%, 1 to 15%, 1 to 10%, 1 to 9%, 1 to 8%, 1 to 7%, 1 to 6%, 1 to 5%, 1 to 2%, 2 to 25%, 2 to 20%, 2 to 15%, 2 to 10%, 2 to 9%, 2 to 8%, 2 to 7%, 2 to 6%, 2 to 5%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v of the inhibitor, the remainder being comprised of water and pharmaceutically acceptable excipients and/or other active agents if being used.

In a further aspect of the invention there is provided an in vitro method for diagnosing WNT/β-catenin dependent cancers, said method comprising
(i) contacting a sample of cells from a test cancer with one or more of the WNT/β-catenin signal transduction inhibitors disclosed herein, and
(ii) assessing the effects of said inhibitor on said sample

Such effects may be one or more selected from level of WNT/β-catenin signal transduction, cell size, replication rate, asymmetric cell division, cancer morphology, respiration, levels of cancer biomarkers. The cells may be cultured in a soft agar colony assay, e.g. as described in the Examples. Alternatively or additionally, an organoid system may be used.

The invention will be further described with reference to the following non-limiting Examples in which:

FIG. 1 shows molecular structures of a: axitinib; b: pazopanib; c: orlistat; and d: topotecan.

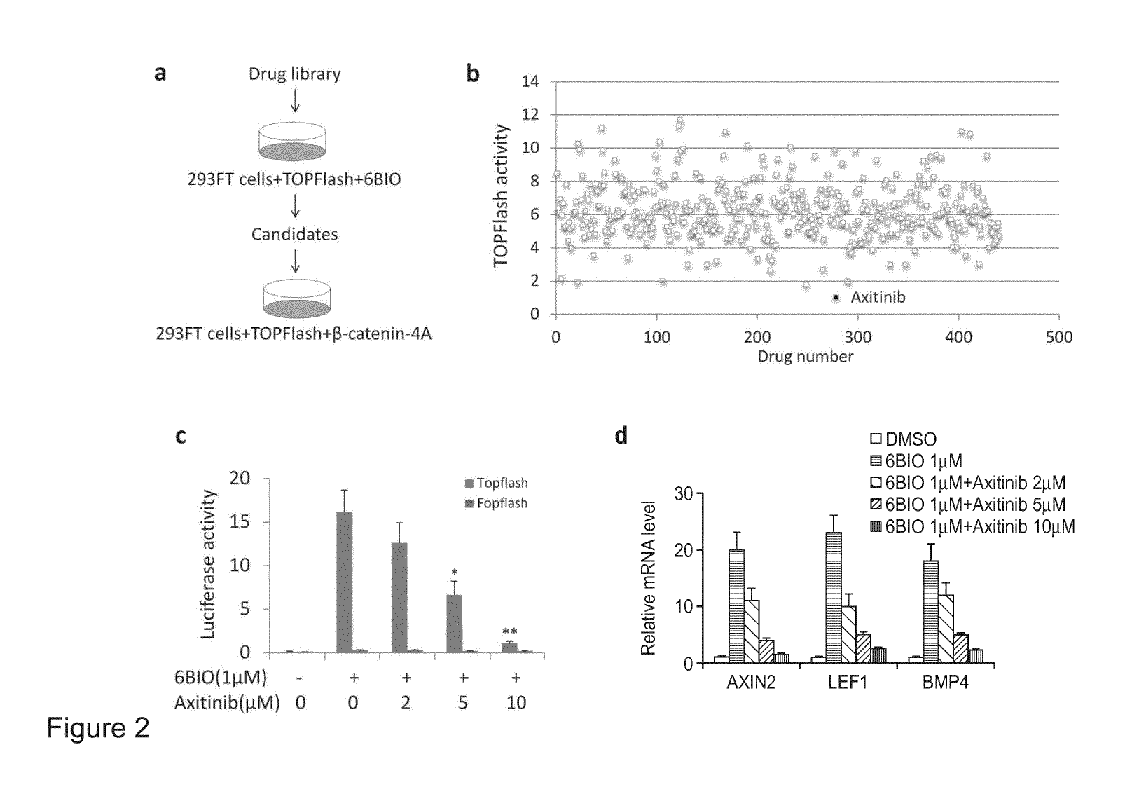

FIG. 2 shows a summary of the screen of FDA approved drugs inhibiting Wnt/β-catenin signaling. a. Schematic diagram of the Wnt inhibitor screening strategy. Wnt/β-catenin signaling is activated by treatment with the GSK3β inhibitor 6BIO in 293FT cells and measured by TOPFlash reporter. Candidates are further tested in 293FT cells overexpressing β-catenin 4A that is resistant to the AXIN/GSK313/APC destruction complex. b. TOPFlash assay of an FDA approved drug library. 293FT cells were treated with 6BIO (1 μM) and 460 FDA approved drugs (10 μM) for 24 hours before TOPFlash assay. Data present TOPFlash activity normalized to Renilla luciferase activity. The drug showing strongest inhibition of TOPFlash activity is indicated. c. Axitinib dose dependently inhibits TOPFlash activated by 6BIO in 293FT cells. A FOPFlash reporter with mutant TCF binding sites was used as negative control. Graph presents mean (s.d.) of TOPFlash or FOPFlash activity normalized to Renilla luciferase activity. * p<0.05, **p<0.01. d. Axitinib decreases mRNA levels of Wnt target genes. 293FT cells were treated as indicated for 24 hours before harvesting for total RNA purification and RT-PCR. Data shown represent mean (s.d.) of relative mRNA expression of indicated genes in triplicated real-time PCR reactions.

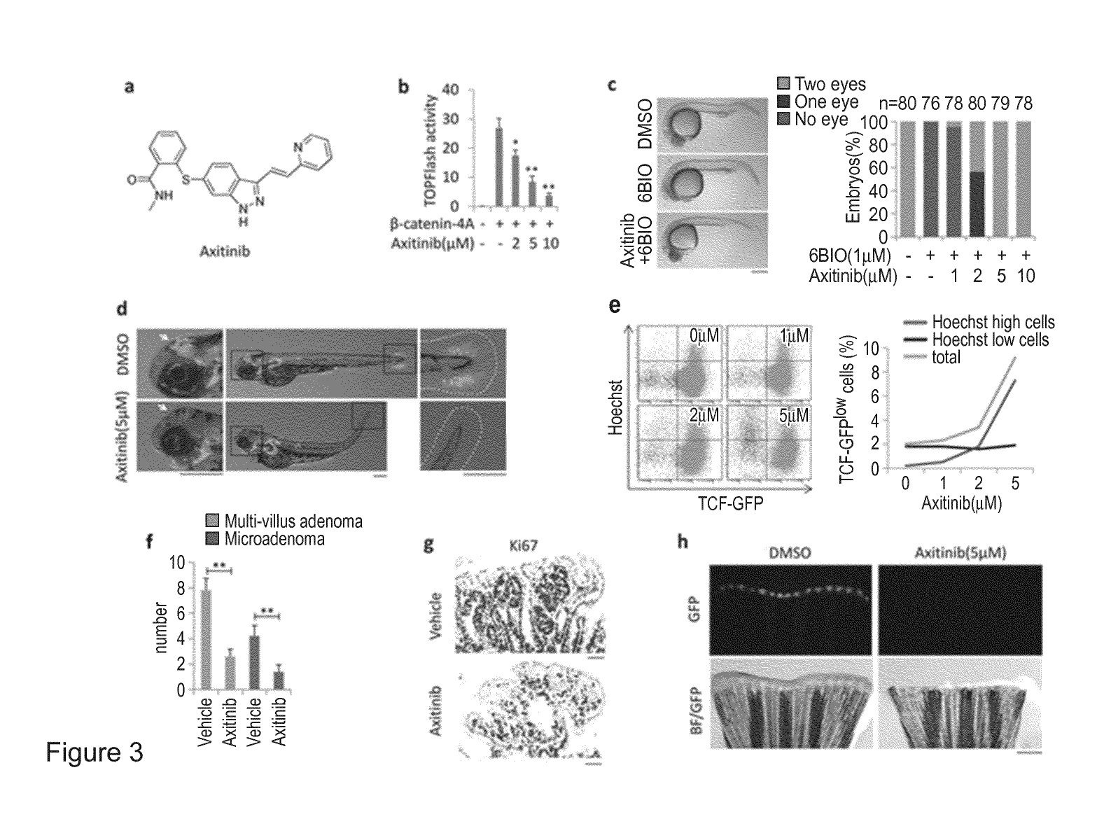

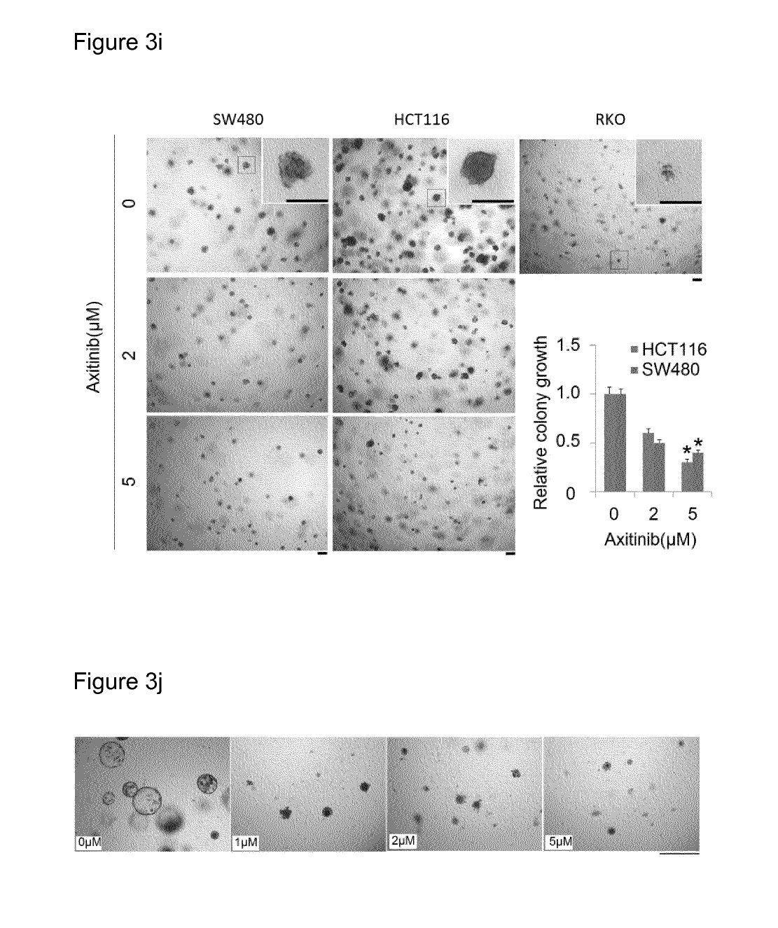

FIG. 3 shows that axitinib inhibits Wnt/β-catenin signaling in vitro and in vivo. a. Chemical structure of axitinib. b. TOPFlash assay of 293FT cells transfected and treated as indicated for 24 hours, data represent TOPFlash activities of three experiments (mean±s.d.), *p<0.05, **p<0.01. c. Representative images of eyeless phenotype rescue assay. Zebrafish embryos at 6 hours post fertilization (hpf) were treated as indicated and the eyeless phenotype was assessed 24 hours later. n=total number of embryos over 4 independent experiments. d. Representative images of TCF-GFP transgenic zebrafish embryos (28 hpf, 40 of each group) treated as indicated for 2 days. TCF-GFP expression in the middle-hindbrain boundary (left, white arrows) and caudal fin mesenchyme (right) are enlarged. e. Flow cytometry analysis of SW480-7TGC cells treated with axitinib for 24 hours followed by Hoechst33342 (10 μM) incubation for 20 minutes. Graph represents the changes of TCF-GFP^{low} cells with different Hoechst intensities, high Hoechst intensity indicates apoptosis. f. Small intestinal adenomas in Apc^{min/+} mice treated with vehicle or axitinib (5 mice per group, **p<0.01). g. Representative immunohistochemistry (IHC) staining of Ki67 in adenomas of Apc^{min/+} mice treated as indicated. h. Representative images of tailfin regeneration of TCF-GFP transgenic zebrafish (13 weeks, 5 fish per group) treated as indicated for 6 days. i. SW480, HCT116 and RKO cells were seeded in soft agar and treated with axitinib at indicated concentrations and imaged 10 days later. Colony growth was quantified by measurement of OD590 and graph presents the OD590 ratios in triplicated experiments. j. Representative images of Apc mutant organoids treated with axitinib. Mouse Apc (VilCre^{ER} Apc^{fl/fl}) intestinal organoids were seeded in Matrigel and treated with axitinib at indicated concentrations for 7 days before imaging. Scale bars: c, d, h, 200 μm, g, i, j, 100 μm.

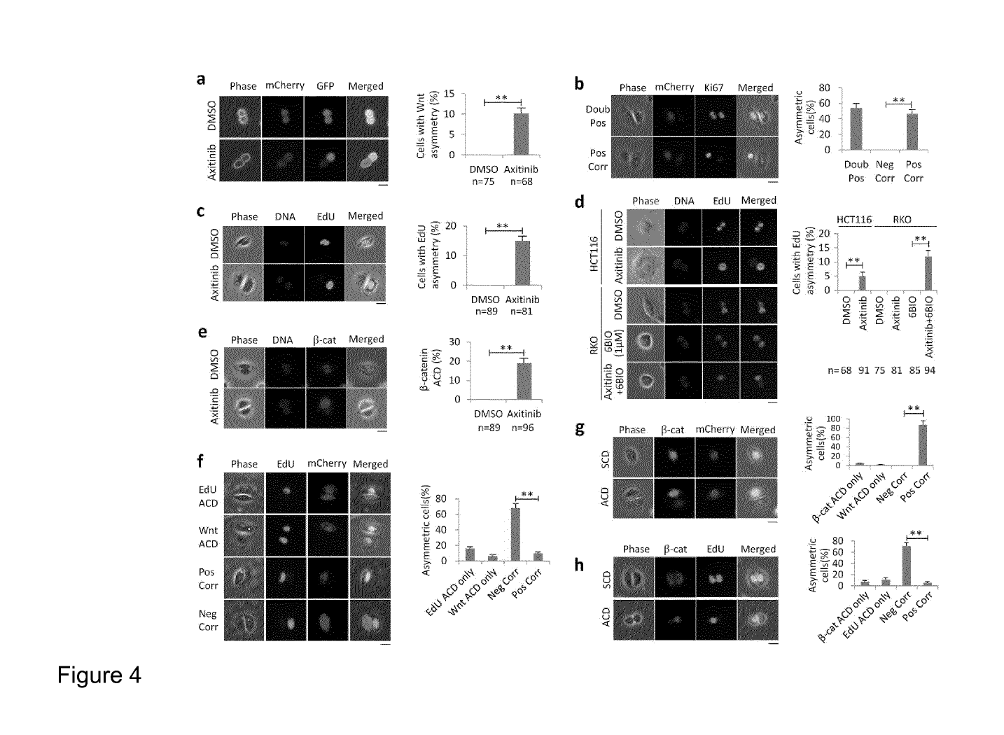

FIG. 4 shows that axitinib directs asymmetric cell division. All the cells were treated with DMSO or 5 μM axitinib for 24 hours unless noted otherwise, n=total counted paired cells over 3 independent experiments. a. Microscope images and quantitation of paired SW480-7TGC cells with unequal TCF-GFP expression. b. Immunofluorescence staining of Ki67 in axitinib treated SW480-7TC cells with unequal TCF-mCherry expression (n=170). Doub Pos, Neg corr and Pos Corr mean positive of Ki67 in both cells, only in Wnt low cell or high cell, respectively. c. EdU label release assay of SW480 cells treated as indicated for 3 days. Paired cells with EdU labeling were scored. d. EdU label release assay of HCT116 and RKO cells treated as indicated for 3 days. e. Immunofluorescence staining of β-catenin (β-cat) in SW480 cells and quantitation of paired cells with unequal β-catenin. f. Correlation of unequal TCF-mCherry (Wnt ACD) and non-random DNA segregation (EdU ACD) in axitinib treated SW480-7TC cells (n=195). g, Correlation of unequal β-catenin (β-cat ACD) to Wnt ACD in axitinib treated SW480-7TC cells (n=134). h. Correlation of β-cat ACD to EdU ACD in SW480 cells treated with 5 μM axitinib for 3 days (n=212). **p<0.01. Scale bars: 20 μm.

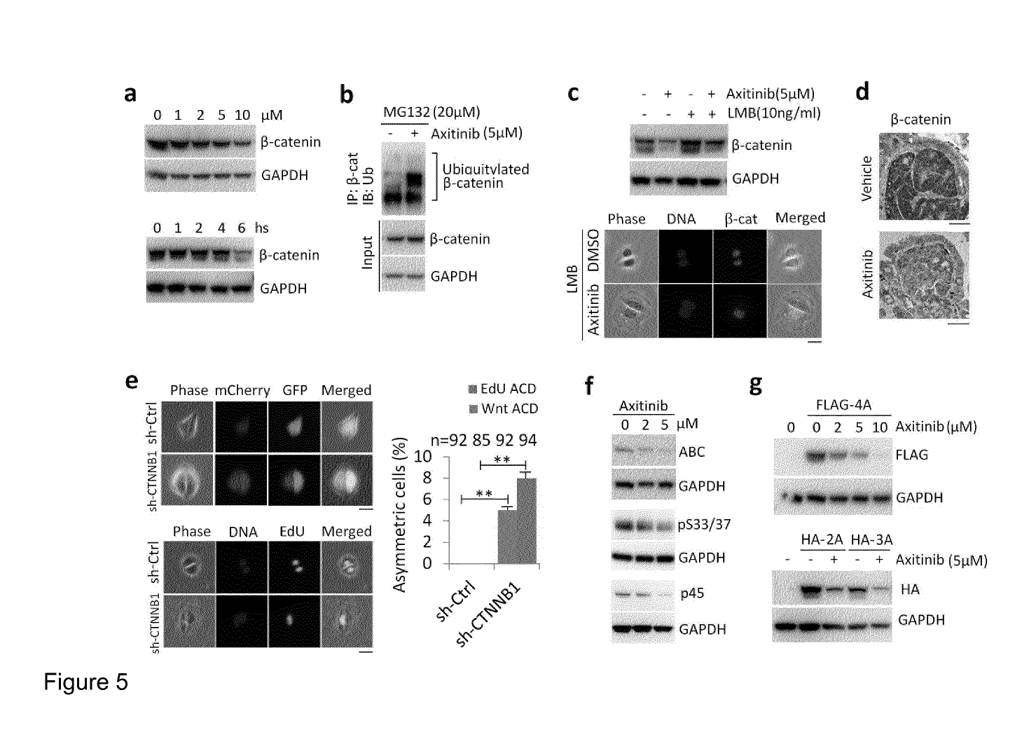

FIG. 5 shows that axitinib promotes nuclear β-catenin degradation. a. Western blots of SW480 cells treated with axitinib in a dose course for 24 hours (up) and time course at 5 μM (down) using antibodies against β-catenin and GAPDH (load control). b. In vivo ubiquitination assay of SW480 cells treated with MG132 together with DMSO or axitinib for 6 hours. c. SW480 cells treated with nuclear export inhibitor leptomycin B (LMB) together with DMSO or axitinib for 24 hours were analyzed by immunoblotting (up) and immunofluorescence staining (down). d. Representative IHC staining of β-catenin in intestinal adenomas of Apc^{min/+} mice described in FIG. 1f. e. Knockdown of CTNNB1 induced Wnt ACD in SW480-TGC cells (up) and EdU ACD in wild type SW480 cells (down). **p<0.01. f. Western blots of SW480 cells treated as indicated for 24 hours using antibodies against non-phosphorylated β-catenin (ABC), β-catenin phosphorylated at Ser33/Ser37 (pS33/37) or Ser45 (pS45) and GAPDH. g. Western blots of 293FT cells transfected with FLAG or hemagglutinin (HA) tagged β-catenin mutants (4A, 2A, 3A) and treated with axitinib as indicated for 24 hours. 4A: Ser33A/Ser37 A/Thr41A/Ser45A, 2A:W504A/1507A, 3A:W504A/1507A/S715A. Scale bar: b, 100 μm, c, d, 20 μm.

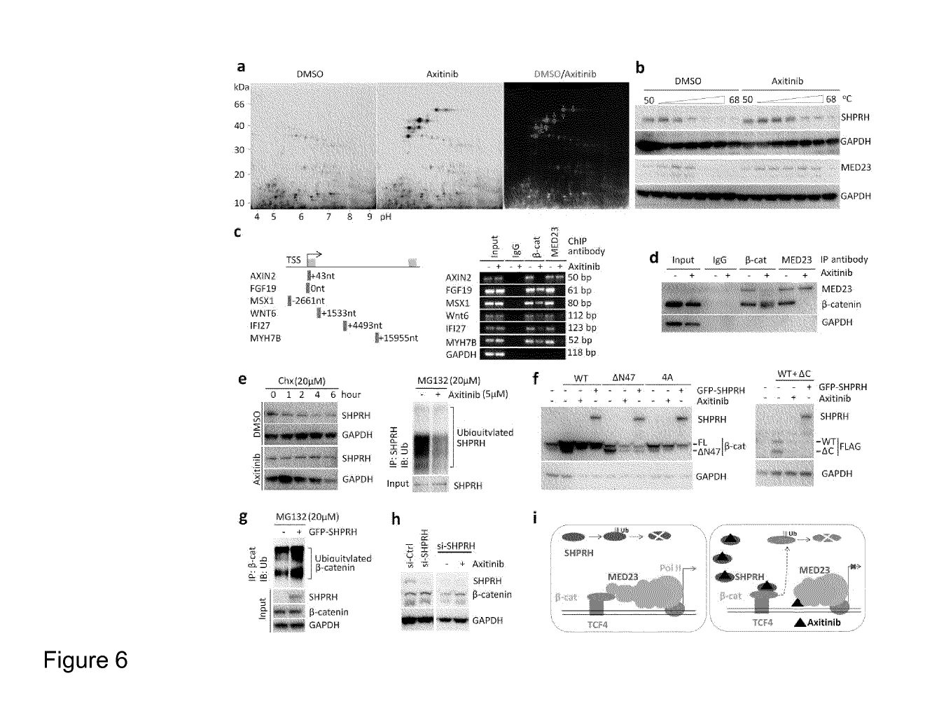

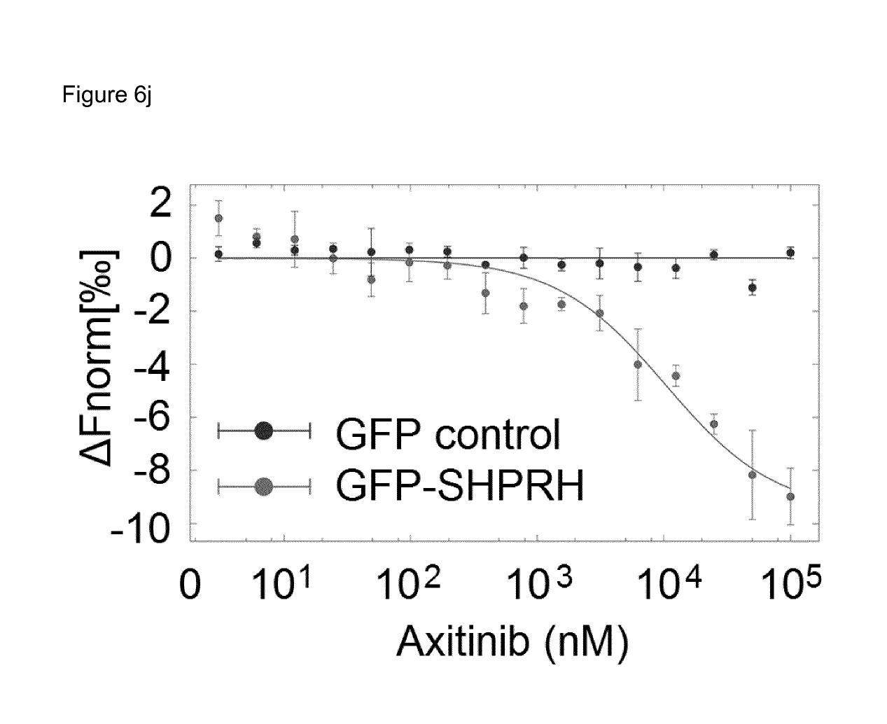

FIG. 6 shows that axitinib blocks Wnt/β-catenin signaling by targeting MED23 and SHPRH. a. 2D-DIGE images of the DARTS samples. SW480 protein lysates were incubated with axitinib (150 μM) or DMSO and digested with pronase before 2D-DIGE. b. Western blots of soluble proteins of SW480 cells incubated with axitinib (10 μM) or DMSO for 2 hours at 37° C. followed by heating at indicated temperatures. c. Location of β-catenin binding motifs of indicated genes (left) and agarose gel analysis of ChIP-PCR reactions with indicated antibodies (right). d. Western blots of the CoIP samples of SW480 cells using indicated antibodies. e. Western blots of SW480 cells treated with DMSO or axitinib (5 μM) for 6 hours together with cycloheximide (Chx) at indicated times (left) and MG132 (right) for 6 hours. f. Overexpression of GFP-SHPRH or axitinib treatment in SW480 cells reduced FLAG tagged β-catenin with indicated mutations. WT, wild type; FL, full length. g. Overexpression of SHPRH in SW480 cells increases the ubiquitination level of β-catenin. h. Knockdown of SHPRH blocked the reduction of β-catenin by axitinib in SW480 cells. i. Summary of the mechanisms by which axitinib blocks Wnt/β-catenin signaling. In the nucleus, treatment by axitinib increases the stability of SHPRH and dissociates the interaction between MED23 and β-catenin. j. Microscale thermophoresis (MST) analysis of axitinib binding to GFP-SHPRH (4 replicates) or free GFP (negative control; 2 replicates) in SW480 cell lysates. The fitted binding curve gives a Kd of 10.4±3.3 μM.

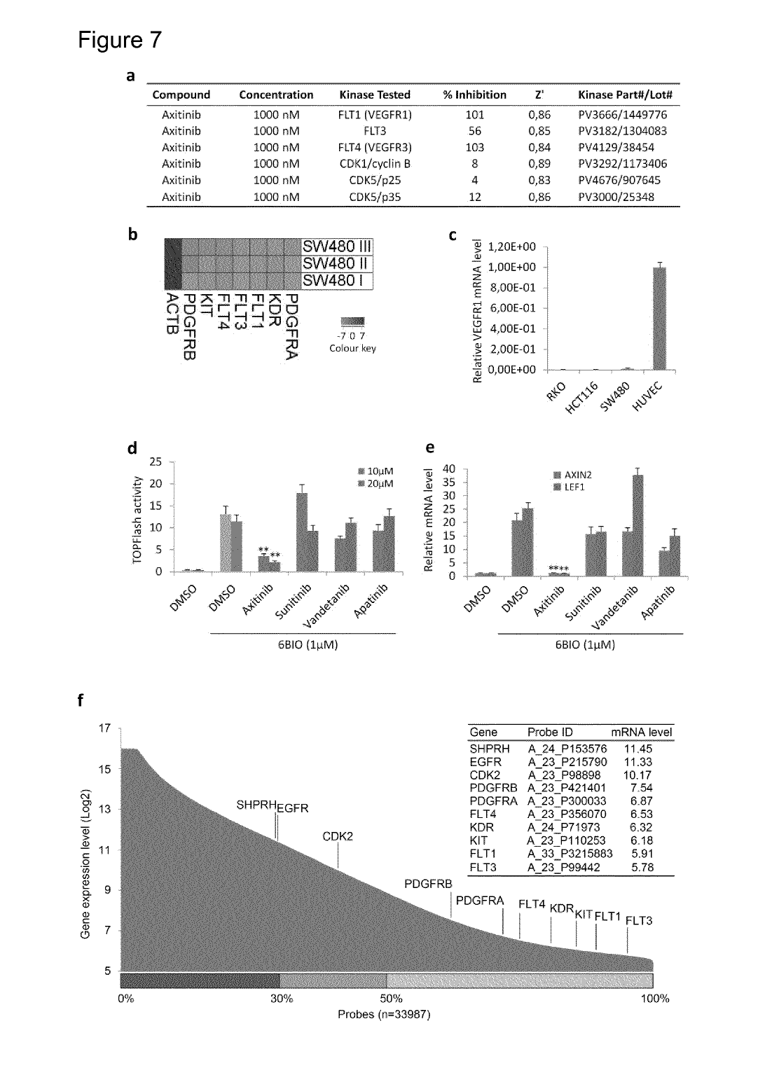

FIG. 7 shows that axitinib inhibits Wnt/β-catenin independently of VEGFRs. a. In vitro enzyme activity assays. Axitinib inhibition of VEGFR1, 3 and FLT3 was determined by using the Adapta Universal Kinase Assay. Kinases CDK1 and CDK5 were used as negative controls. b. DNA microarray profiling of SW480 cells shows the mRNA levels of β-actin (ACTB) and genes coding potential target of axitinib. FLT1, KDR and FLT4 are known as VEGFR1, 2 and 3, respectively. c. Quantitative RT-PCR analysis of VEGFR1 mRNA level in indicated cell lines. HUVEC cells were used as positive control. d. TOPFlash assay of VEGFR inhibitors. 293FT cells were treated with 6BIO (1 μM) together with DMSO or VEGFR inhibitor Sunitinib, Vandetanib and Apatinib for 24 hours. Axitinib was used as positive control. e. RT-PCR analysis of the expression of Wnt target gene AXIN2 and LEF1. 293FT cells were treated as indicated for 24 hours before total RNA purification. **p<0.01. f. DNA microarray profiling of SW480 cells and ranking of the genes based on the expression levels. Gene expression value (log 2) was generated and normalized by the J-express program; the highest and lowest log 2 value was 16 and 5, respectively. The top 50% and 30% genes were considered as expressed and highly expressed genes, respectively. The expression of axitinib known kinase targets (FLT1, KDR, FLT4, KIT, FLT3, PDGFRA, PDGFRB), kinases known to be present in all cycling cells (CDK2, EGFR) and SHPRH are indicated.

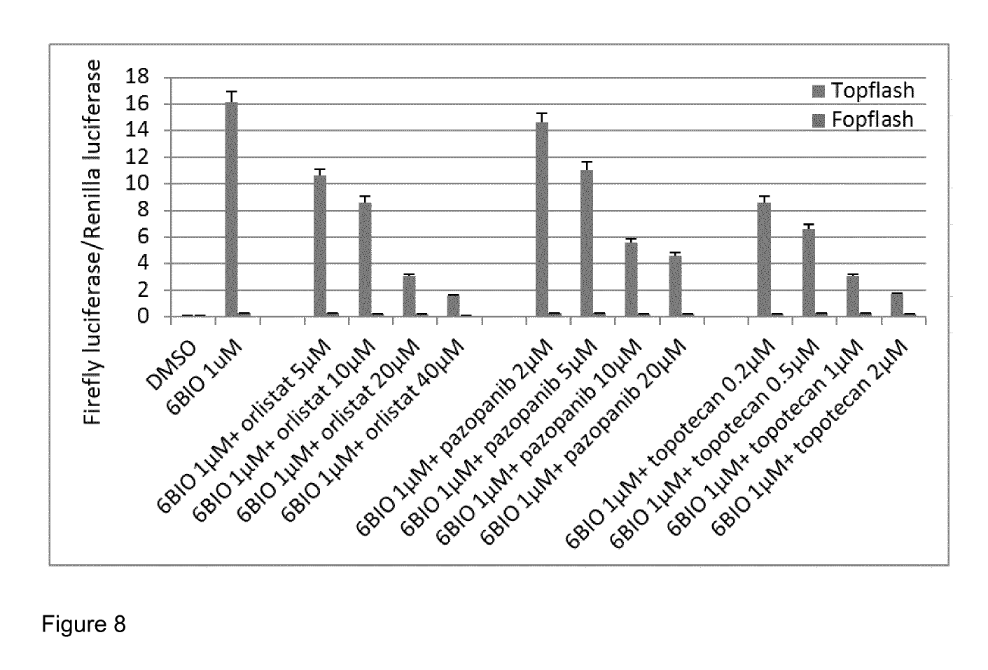

FIG. 8 shows Wnt signaling inhibition by pazopanib, orlistat and topotecan using the Topflash assay. 293FT cells transfected with 0.22 ug TOPFlash (or FOPFlash with mutant TCF binding sites) were treated with DMSO, 1 μM 6BIO and 1 μM 6BIO together with pazopanib, orlistat and topotecan at indicated concentrations for 24 hours and then luciferase activity was measured. Graph presents mean (s.d.) of TOPFlash or FOPFlash activity normalized to Renilla luciferase activity.

### EXAMPLES

### Example 1—Axitinib Blocks Wnt/β-Catenin Signaling and Directs Asymmetric Cell Division in Cancer

Introduction

Cancer genome sequencing has revealed APC and CTNNB1 as mutated genes in certain cancer types, and most of the mutations are predicted to activate oncogenic Wnt/β-catenin signaling. Although substantial efforts have been invested, there is still a lack of therapeutic agents blocking the Wnt/β-catenin pathway, especially downstream of APC and β-catenin. Here we show that the FDA approved drug axitinib inhibits Wnt-dependent processes in zebrafish and tumor growth in Apc mutant mice with minor effect on adult tissue homeostasis. In APC mutant cancer cells, axitinib dramatically directs asymmetric cell division in terms of unequal Wnt signaling and non-random DNA segregation by promoting proteasome degradation of nuclear β-catenin independent of the GSK313/APC complex. Using chemical proteomics approaches, we identify the mediator complex subunit MED23 and E3 ubiquitin ligase SHPRH as major direct target proteins of axitinib in blocking Wnt/β-catenin signaling. Treatment of axitinib dissociates MED23 from β-catenin and Wnt target genes, while binding of axitinib stabilizes SHPRH which increases ubiquitination and degradation of β-catenin. Our findings suggest that axitinib, as a clinically approved drug, would provide therapeutic benefits for cancer patients with Wnt pathway mutations and also other diseases or conditions in which WNT/β-catenin signal transduction is a contributing factor.

Materials and Methods

Chemicals

An FDA Approved Drug Screening Library (L1300), axitinib (S1005), Sunitinib (S1042), Vandetanib (S1046) and Apatinib (S2221) were products of Selleckchem, 5-Bromo-2′-deoxyuridine (B5002), thymidine (T9250), MG132 (M7449), leptomycin B (L2913), cycloheximide (C4859), Hoechst 33342 (B2261), Disuccinimidyl suberate (S1885), Disuccinimidyl glutarate (80424), N-Ethylmaleimide crystalline (04259) and Ethylene glycol-bis (E3257) were purchased from Sigma, 6B10 (ALX-430-156-M001) was purchased from Enzolifesciences.

Plasmids and siRNAs

Super 8×TOPFlash (12456), Super 8×FOPFlash (TOPFlash mutant) (12457), pRL-SV40P (27163), 7×Tcf-eGFPHSV40-mCherry (7TGC) (24304), pcDNA3-3-catenin (16828), β-catenin-ΔN47 (19287), β-catenin-4A (S33A/S37A/T41A/S45A) (24204), sh-CTNNB1-1248 and sh-CTNNB1-2279, pMD2.G (12259) and pCMVR8.74 (22036) were obtained from Addgene (Cambridge, Mass.). Lentivirus shRNA vectors for MED23 were gifts from Dr. Michael Carey at UCLA, USA. MED23 expression construct pCR3-MED23 was a gift from Dr. Jurgen Haas at University of Edinburgh, UK. HA tagged β-catenin-2A (ΔLIR) containing mutation W504A/1507A was a gift from Dr. Alex Greenhough at University of Bristol, UK. GFP tagged SHPRH vector was a gift from Dr. Karlene Cimprich at Stanford University, USA. Stealth siRNAs for SHPRH (HSS138073, HSS138074 and HSS138075) and negative control were products of Life Technologies.

QuickChange Multiple Site-directed Mutagenesis kit (Stratagene, La Jolla, Calif.) was used to introduce mutations to plasmids. To generate a TCF-mCherry Wnt reporter (7TC), two BamHI cut sites (GGATCC) were introduced to the 7TGC vector by using mutant PCR primers 5′-CCTTGCTCACCATGGATCCTTTACCAACAGTACCGG-3′ and 5′-CGCCCTTGCTCACCATGGATCCCTTTTTGCAAAAGCCTAGGCC-3′. The mutated 7TGC vector was digested by BamHI to remove an 1180 bp fragment containing EGFP and SV40 promoter, the remained vector was ligated to form a TCF-mCherry cassette. To generate a C-terminal truncated β-catenin (AC), residue Q668 was mutated to a stop code by using PCR primer: 5′-GTCTGAGGACAAGCCATAAGATTACAAGAAACGGCTTTCAGTTG-3′. To generate β-catenin-5715A, the following oligo was used for mutant PCR: 5′-TGGATATCGCCAGGATGATCCTGCAGATCGTTCTTTTCACTCTGG-3′.

Cell Lines, Cell Culture, Cell Transfection and Lentiviral Transduction

SW480 (CCL-228), HCT116 (CCL-247) and RKO (CRL-2577) cells were obtained from American Type Culture Collection (ATCC) and maintained according to the supplier's recommendations. 293FT cells were purchased from Life Technologies. Human umbilical vein endothelial cells (HUVEC) were purchased from Lonza. Leibovitz's L-15 medium for SW480 cells, McCoy's 5A medium for HCT116 cells, Eagle's minimum essential medium for RKO cells, Dulbecco's modified eagle's medium for 293FT and EGM-plus growth medium for HUVEC cells were purchased from Lonza. Prostate EPT3 cells were established in our lab and the culture methods have been described previously (Ke, X. S. et al, 2008, PLoS One 3, e3368; Qu, Y. et al. Cancer Res 73, 2013, 7090-7100). All cell lines have been authenticated by DNA microsatellite fingerprinting³¹, and the mycoplasma contamination was ruled out using the MycoAlert™ Mycoplasma Detection Kit (Lonza).

For DNA and siRNA transient transfection, cells were transfected using lipofectamine 3000 and Lipofectamine® RNAiMAX (Life Technologies), respectively. To establish stable cell lines containing Wnt reporters, lentiviral vector 7TGC or 7TC together with packaging plasmids pMD2.G and pCMVR8.74 were co-transfected into 293FT cells with lipofectamine 3000. After 16 hours, culture medium was replaced with medium for cells of interest. One day later, the culture medium was filtered through a 0.45 μm filter and incubated with cells for 24 hours. The transduced cells were assessed by fluorescence microscopy and fluorescence-activated cell sorting (FACS Aria, BD Biosciences).

TOPFlash Assay

TOPFlash assay was performed in 96-well plate. For each well, 293T cells were transfected with 0.22 μg TOPFlash (or FOPFlash with mutant TCF binding sites) and 0.02 μg pRL-SV40P using lipofectamine 3000. For co-transfection with other plasmids, 0.1 μg additional DNA was used. Four hours after transfection, 293FT cells were treated with 1 μM 6B10 and 1 μM 6B10 together with axitinib at varying concentrations for 24 hours. The luciferase activity was measured 24 hours later by using Dual-Glo® Luciferase Assay System (Promega, E2940) according to the recommended protocol. The TOPFlash or FOPFlash activity was normalized to Renilla luciferase signals.

Zebrafish Study

Transgenic zebrafish harboring Tcf/Lef-miniP:dGFP reporter (line isi04) was obtained from National BioResource Project Zebrafish, Japan. Fish were kept under standard conditions and treated humanely in accordance with approved Institutional Animal Care and Use Committee of University of Bergen. For embryo experiments, embryos at 6 hpf (hours post fertilization) were cultured in 6-well plates (20 embryos per well) at 28° C. Embryos were treated with indicated chemicals and water was replenished daily. For eyeless phenotype rescue assay, embryos at 2 days post fertilization (dpf) were scored for the eye development (two eyes, one eye or no eye). For TCF-GFP expression assay, embryos at 3 dpf were examined under fluorescence microscope. In both assays embryos that died during treatment were excluded from assessment.

For tailfin regeneration assay, transgenic zebrafish at 3 months were placed in a beaker of diluted Tricain solution (0.1 g in 500 ml fish water or E3) until fully anesthetized (1 to 2 minutes). A single vertical cut perpendicular to the rays of the fin was made by dissecting scissors. A total of 10 amputees were randomly divided into two groups with similar average length, and reared at 28° C. in tanks containing either 5 μM axitinib or the same volume of DMSO, respectively. Water and compounds were replenished daily for a period of 6 days, the length of the tail was measured by ruler when they were anesthetized every day to evaluate the tail regeneration, TCF-GFP expression in the tail was examined under a fluorescence microscope. For BrdU incorporation study, at the end of tailfin regeneration assay, zebrafish were incubated with BrdU (1 mM) for 2 hours at 28° C. before anesthesia. The gastrointestinal tract was fixed in 4% (v/v) paraformaldehyde, paraffin embedded and sectioned. Sections were stained with hematoxylin and eosin or processed for BrdU immunohistochemistry by following Abcam′ BrdU staining protocol.

Apc^{min/+} Mice Study

C57BL/6-Apc^{min/+} mice were obtained from the Model Animal Research Center of Nanjing University (Nanjing, China). Mice were housed and fed a standard rodent diet at the Animal Facility of the Second Military Medical University (Shanghai, China) in compliance with the institutional guidelines of the Animal Care and Use Committee. After 1 week of acclimation, a total of 10 Apc^{min/+} male mice (7 weeks of age) were randomly divided into two groups with similar average in weight. Mice were administered with vehicle control (0.5% carboxymethylcellulose/H₂O.HCl (pH 2-3)) or axitinib at 50 mg/kg, respectively, by oral gavage daily for 5 consecutive weeks. The mice were weighed weekly and monitored daily for any signs of illness. The mice were sacrificed at the last day of the treatment. The small intestines were dissected, washed in PBS, fixed in 4% PBS-buffered formaldehyde and embedded in paraffin using standard procedures.

Soft Agar Colony-Formation Assay and Clonogenic Assay.

The soft agar colony-formation assay was performed using the CellTransformation Assays kit (catalog no. CBA-130; Cell Biolabs, Inc.) according to the recommended protocol. SW480, HCT116, and RKO cells were assayed in 96-well plates and were treated with axitinib at the indicated concentrations. Medium containing drugs was replaced every 3 d. For the clonogenic assay, positively transfected cells (wild-type and mutant GFP-SHPRH and control GFP) were enriched by FACS and were seeded in six-well plates at a density of 3,000 cells per well. Medium was changed every 3 d. On the last day colony growth was quantified by crystal violet staining and measurement of OD at 590 nm.

Organoid Culture.

Mouse intestinal Apc (VilCre^{ER} Apc^{fl/fl}) organoids were obtained from Owen J. Sansom's laboratory at the Beatson Institute for Cancer Research, Glasgow, UK. The culture protocol has been described previously (14). Briefly, Apc mutant organoids were suspended in Growth Factor Reduced Matrigel (catalog no. 356231; Corning) and cultured in Advanced DMEM/F12 (catalog no. 12634-028; Invitrogen) containing 1% B-27 supplement (50×), minus vitamin A (catalog no. 12587-010; Invitrogen) and 0.1% BSA. Organoids were seeded in 24-well plates at a density of 70-100 organoids/50 μL Matrigel in each well. One day later DMSO or axitinib at the indicated concentration was added; medium was replaced every 3 d. One week later organoids were imaged using the Cytation 5 Cell Imaging Multi-Mode Reader (BioTek Instruments, Inc.).

Hematoxylin and Eosin (H&E) and Immunohistochemistry (IHC) Staining

H&E and IHC were performed according to the protocol previously described (Qu, Y. et al, supra). Primary antibodies used for IHC were β-catenin (ab16051, 1:1000, Abcam), Ki67 (ab16667, clone SP6, 1:100, Abcam) and BrdU (ab6326, clone BU1/75, 1:80, Abcam). Investigators were blinded when counting the intestinal adenomas and assessing the homeostasis of mice and fish intestine. Histologic images were captured using the Qcapture Suite software with a Qimaging Exi Blue camera attached to a Leica DMRBE microscope.

Wnt ACD Assay

Cells containing 7TGC or 7TC reporter were synchronized to G1/S phase by double thymidine block (18 hours exposure-9 hours release-15 hours exposure) and plated singly to new plates, DMSO or axitinib (5 μM) was added immediately after seeding, 16 hours later the images were captured and the intensity of TCF-GFP or TCF-mCherry in paired cells were quantified by Photoshop CS6 software. Unequal Wnt signaling was considered when paired cells had intensity ratio higher than 2.

Live Cell Imaging

Live cell imaging was done by using a Cytation 3 Cell Imaging Multi-Mode Reader (BioTek Instruments, Inc., USA). SW480-7TGC cells were synchronized to G1/S phase and seeded singly in 96-well black plate. After 3 hours in the incubator, cells were kept in the reader at 37° C. for a time-period of 2 hours with an imaging step every 5 minutes. Images were acquired in the GFP channel (ex 469/35, em 525/39) and bright-field channel using a 10× objective. Data were visualized and analyzed with the Biotek Gen5_ver2.06 software.

Immunofluorescence

Cells were seeded singly on 12 mm glass coverslips in 24-well plates. At the second day, cells were washed with PBS, fixed in 4% PBS buffered paraformaldehyde at room temperature for 20 minutes, permeabilized in 0.5% Triton X-100 for 10 minutes, blocked in 100 mM glycine for 5 minutes and with PBS wash between each step. After blocking with 0.5% BSA/PBS for 15 minutes, cells were incubated with primary antibodies (β-catenin, ab16051, 1:1000, Abcam; Ki67, ab16667, clone SP6, 1:50, Abcam) in 0.5% BSA/PBS for 1 hour at room temperature. The FITC-labelled secondary antibody (Southern Biotech, 4050-02) was added for 45 minutes at room temperature. Coverslips were mounted in SlowFade® Diamond Antifade Mountant with DAPI (Life Technologies, S36964) on glass slides. Images were captured using the Qcapture Suite software with a Qimaging Exi Blue camera attached to a Leica DMRBE microscope.

EdU Label Release Assay

Culture cells were treated with 5 μM axitinib/DMSO or transfected with sh-CTNNB1/sh-control plasmids for 24 hours before incubation with 10 μM EdU at 37° C. for 30 minutes. Cells were washed with PBS intensively and plated singly to new plates containing 5 μM axitinib or DMSO. Two days later, cells were seeded singly onto 12 mm glass coverslips in 24-well plates. One day after, EdU was detected using the Click-iT® Plus EdU Alexa Fluor 488 Imaging Kit (Life Technologies, C10637) according to the manufacturer's protocol. For double staining of EdU and other proteins, the EdU stained cells were further used for immunofluorescence staining as described above. Intensity of EdU staining in paired cells were quantified by Photoshop CS6 software, unequal EdU distribution was considered when paired cells had intensity ratio higher than 2.

In Vitro Kinase Assay

Inhibition by axitinib of VEGFR1, 3 and FLT3 was determined by SelectScreen Kinase Profiling Service using the Adapta Universal Kinase Assay (Life Technologies, Paisley, UK). Kinases CDK1 and CDK5 were used as negative controls. Assays were performed using 1 μM Axtinib for all kinases.

DNA Microarray

Genome wide transcription profiling using Agilent DNA microarray 44k (Agilent Technologies, G4112F and G4845A) has been described previously (Qu, Y. et al, supra). Raw data were imported and analyzed in J-Express software (Molmine, http://www.molmine.com). Mean spot signals were used as intensity measure, the expression data were quantile normalized over the entire arrays and log 2-transformed. Differentially expressed genes were identified using the feature subset selection (FSS) method.

Western Blotting

Culture cells were lysed in RIPA buffer (50 mM Tris-HCl, pH 7.4, 150 mM NaCl, 1% NP-40, 0.5% sodium deoxycholate, 0.1% SDS, 1 mM EDTA) containing freshly added protease inhibitors (Roche, 11836153001). Protein lysates were resolved in Novex 10% BisTris MiniGels (Life technologies, NP0303BOX) and transferred onto PVDF membranes with Pierce 1-Step Transfer Buffer (Thermo Fisher Scientific, 84731). Membranes were developed by using Pierce Fast Western Blot Kit with ECL Substrate (Thermo Fisher Scientific, 35050) and primary antibodies (β-catenin, ab16051, 1:2000 Abcam; GAPDH, ab181602, clone EPR16891, 1:20000, Abcam; hemagglutinin, ab9134, 1:2000, Abcam; FLAG, ab1162, 1:1000, Abcam; ubiquitin, ab7254, clone Ubi-1, 1:5000, Abcam; SHPRH, ab80129, 1:1000, Abcam; non-phospho-(active) β-catenin (Ser33/37/Thr41), 4270, 1:1000, Cell Signaling Technology; phospho-3-catenin (Ser33/37/Thr41), 9561, 1:1000, Cell Signaling Technology; β-catenin-Thr41/Ser45, 9565, 1:1000, Cell Signaling Technology; MED23, BD550429, clone D27-1805, 1:500, BD Biosciences). Proteins were visualized and captured by a ChemiDOC XRS system and Quantity One software (Bio-Rad Laboratories).

Drug Affinity Responsive Target Stability (DARTS) Assay

DARTS assay was performed according to the protocol previously described (Lomenick, B. et al., 2009, Proc Natl Acad Sci USA 106, 21984-21989). Briefly, 1×10⁷ SW480 cells were lysed in 2.4 ml M-PER buffer (Pierce, 78501) containing freshly added protease inhibitors. Cell lysates were centrifuged at 18,000 g for 10 min at 4° C. The supernatant was transferred to a new tube containing 10×TNC buffer ((500 mM Tris-HCl (pH8.0), 500 mM NaCl, 100 mM CaCl₂)), and equally divided between two tubes for incubation 1 hour at room temperature with 20 ul DMSO or axitinib (10 mM), respectively. Incubated samples were proteolyzed with 4.2 mg/ml pronase (Roche, 10165921001) at room temperature for 30 minutes. Digestion was stopped by adding protease inhibitors (Roche, 11836153001) and samples were stored at −80° C. for proteomics analysis.

Two-Dimensional Difference Gel Electrophoresis (2D-DIGE) and Mass Spectrometry

For 2D-DIGE, 10 volumes of 2-D cell lysis buffer (30 mM Tris-HCl, pH 8.8, containing 7M urea, 2M thiourea and 4% CHAPS) were added to the DARTS samples and the original buffer was replaced using 3 kDa MWCO spin columns. Samples treated with DMSO and axitinib were labeled with Cy3 and Cy5, respectively. The labeled samples were mixed well before loading into strip holder. IEF and SDS-PAGE were performed following the protocol provided (Amersham BioSciences). Gel image scans were carried out immediately following the SDS-PAGE using Typhoon TRIO (GE Healthcare). The scanned images were then analyzed by Image QuantTL software (GE Healthcare), and then subjected to in-gel analysis and cross-gel analysis using DeCyder software version 6.5 (GE Healthcare). The ratio change of the protein differential expression was obtained from in-gel DeCyder software analysis.

The spots of interest were picked up by Ettan Spot Picker (GE Healthcare) and digested in-gel with modified porcine trypsin protease (Trypsin Gold, Promega). To identify the peptides, MALDI-TOF (MS) and TOF/TOF (tandem MS/MS) were performed on a 5800 mass spectrometer (AB Sciex). MALDI-TOF mass spectra were acquired in reflectron positive ion mode, averaging 2000 laser shots per spectrum. TOF/TOF tandem MS fragmentation spectra were acquired for each sample, averaging 2000 laser shots per fragmentation spectrum on each of the 5-10 most abundant ions present in each sample (excluding trypsin autolytic peptides and other known background ions). For database search, both the resulting peptide mass and the associated fragmentation spectra were submitted to GPS Explorer version 3.5 equipped with MASCOT search engine (Matrix science) to search the database of National Center for Biotechnology Information non-redundant (NCBInr) or Swiss Protein database. Searches were performed without constraining protein molecular weight or isoelectric point, with variable carbamidomethylation of cysteine and oxidation of methionine residues, and with one missed cleavage allowed in the search parameters. Candidates with either protein score C.I. % or Ion C.I.% greater than 95 were considered significant.

Cellular Thermal Shift Assay (CETSA)

CETSA in intact cells was performed according to the protocol previously described (Martinez Molina, D. et al., 2013, Science 341, 84-87). Briefly, SW480 cells were seeded equally in 2 T75 flasks and allowed to reach 80% confluence one day after. Cells in each flask were incubated with 10 μM axitinib or an equal volume of DMSO respectively at 37° C. for 1 hour. After trypsinization and PBS wash, cells were resuspended in 450 μl PBS containing freshly added protease inhibitors and equally divided between 7 tubes. Cells in each tube were heated at indicated temperatures for 3 minutes and kept at room temperature for 3 minutes. Heated cells were lysed by 3 cycles of freezing in liquid nitrogen (1 minute) and thawing in room temperature water (1 minute). The cell lysates were centrifuged at 20,000 g for 20 minutes at 4° C. The soluble fractions were isolated for Western blotting analysis.

MST Ligand-Binding Assay

MST was used to determine the binding affinity of ligand (axitinib) and receptor [GFP-tagged SHPRH (fusion GFP) or free GFP as control]. Ten million SW480 cells overexpressing GFP-tagged SHPRH or free GFP were lysed in 1 mL radioimmunoprecipitation assay (RIPA) buffer. Cell lysates were diluted in buffer A [50 mM Hepes buffer (pH 7.5), 5 mM DTT, 10 mM CaCl₂), 50 mM NaCl, and 0.05% Tween-20] to a final concentration at which the fluorescent signals of the GFP proteins were similar and well above the detection limit of the Monolith NT.115 instrument (NanoTemper Technologies GmbH). Ten microliters of each receptor were mixed with 10 μL of the ligand at various concentrations from 100 μM to 3.05 nM. Specifically, the ligand (4 mM in 100% DMSO) was diluted 1:20 to a final concentration of 200 μM in buffer A (giving 5% DMSO). Ten microliters of the 200 μM ligand solution was further serially diluted 1:1 in 10 μL of buffer A supplemented with 5% DMSO to make a 16-sample dilution series down to 6.1 nM. Ten microliters of the cell lysate were added to 10 μL of each ligand solution. The GFP-SHPRH-axitinib and GFP-axitinib mixture solutions were loaded into NT.115 standard coated capillaries (NanoTemper Technologies GmbH), and the MST measurements were performed at 25° C., 80% LED power, and 10% IR-laser power. The fluorescence signal during the thermophoresis was monitored for 30 s, and the change in fluorescence was analyzed as thermophoresis with T-jump. The Kd was calculated by fitting a standard binding curve to the average of four independent dilution series. The negative controls (two parallels) did not show any binding of the ligand to free GFP.

In Vivo Ubiquitination Assay

To determine protein ubiquitination in vivo, 5×10⁶ SW480 cells were treated with 20 μM MG132 together with DMSO or 5 μM axitinib for 6 hours. Cells were harvested and re-suspended with 100 μl ice cold TBS (50 mM Tris-HCL pH 8.0, 150 mM NaCl) containing 2 μM N-Ethylmaleimide crystalline that was supplemented to all the following buffers. After adding 120 μl TBS containing 2% SDS and mixing quickly, cells were heated at 98° C. for 10 minutes and placed immediately on ice for 5 minutes. Cell lysates were incubated with 1.8 ml TBS containing 1% Triton X-100 and 3 μg antibody (β-catenin ab22656, clone 12F7, Abcam; SHPRH, ab80129, Abcam), at 4° C. overnight followed by incubation with 100 μl Protein G beads (Life technologies, 10004D) at 4° C. for 1 hour on rotator. Beads were collected with Magnet and serially washed once with 1 ml TBS containing 1% Triton X-100/0.1% SDS, twice with 1 ml TBS containing 0.5M LiCl and once with 1 ml TBS containing 1% Triton X-100. Following addition of 80 μl 2% SDS sample buffer containing 10 mM DTT (Sigma, D9779), the beads were boiled at 98° C. for 5 minutes and collected with Magnet, the supernatant was analyzed by Western blotting.

Co-Immunoprecipitation (Co-IP)

Co-IP was done following the protocol of Dynabeads Co-Immunoprecipitation Kit (Life Technologies, 14321D). Briefly, 600 mg SW480 cells pre-treated with DMSO or 5 μM axitinib for 4 hours were harvested by using 0.25% EDTA free trypsin (Gibco, 15050-065). After saving 2% samples for input control, cell lysates were equally divided between 3 tubes, incubated with 3 mg Dynabeads and 21 μg mouse IgG (15381, Sigma), primary antibody against MED23 (BD550429, clone D27-1805, BD Biosciences) and β-catenin (ab22656, clone 12F7, Abcam), respectively. Input controls and proteins pulled down with individual antibodies were examined by Western blotting.

Chromatin Immunoprecipitation (ChIP)

ChIP was performed according to the protocol previously described with a few modifications on the crosslinking procedures (Ke, X. S. et al., 2009, PLoS One 4, e4687). Briefly, 6×10⁷ SW480 cells pre-treated with DMSO or 5 μM axitinib overnight were cross-linked with protein-protein crosslinkers (0.67 mM Disuccinimidyl suberate, 0.67 mM Disuccinimidyl glutarate and 0.67 mM Ethylene glycol-bis) for 45 minutes at room temperature before the fixation in 0.75% formaldehyde for 10 minutes at 37° C. The reaction was quenched with 1/50 volume of 2.5 M glycine. Cells were lysed and the nuclei were sonicated to fragments ranging from 200 bp to 500 bp. After saving 0.2% total chromatin for input DNA purification, sonicated lysates were equally divided to three tubes and immunoprecipitated with Protein G beads (Life technologies, 10004D) coupled with 5 μg mouse IgG (15381, Sigma), antibody against MED23(BD550429, clone D27-1805, BD Biosciences) and β-catenin (ab22656, clone 12F7, Abcam), respectively. The immunoprecipitated DNA was purified and examined by PCR described below.

Reverse Transcription and Real-Time Quantitative PCR

Reverse transcription (RT) and real-time quantitative PCR (qPCR) were done as previously described (Ke, X. S. et al., 2011, Exp Cell Res 317, 234-247). For qPCR using TagMan Universal PCR Master Mix (Life technologies, 4304437), TagMan assays used for human AXIN2 (Hs00610344_m1), LEF1 (Hs01547250_m1), BMP4 (Hs03676628_s1), CTNNB1 (Hs01076483_m1), VEGFR1 (Hs01052961_m1), MED23 (Hs00606608_m1), SHPRH (Hs00542737_m1) and ACTB1 (Hs99999903_m1) were obtained from Life technologies. For RT-PCR using RT² SYBR Green/ROX PCR Master Mix (QIAGEN, 330520), primer oligos were made by Eurogentec (Liege, Belgium).

FGF19,
forward,
5′-CGCACAGTTTGCTGGAGATCA-3′,
reverse,
5′-CCTCCGAGTACTGAAGCAGCC-3′;
AXIN2,
forward,
5′-GAGGAAGGAGACAGGTCGCAG-3′,
reverse,
5′-TTTGTGCTTTGGGCACTATGGG-3′;
WNT6,
forward,
5′-CTGCAACAGGACATTCGGGA-3′,
reverse,
5′-CAGCTCGCCCATAGAACAGG-3′;
IFI27,
forward,
5′-ATCAGCAGTGACCAGTGTGG-3′,
reverse,
5′-TGGCCACAACTCCTCCAATC-3′;
MYH7B,
forward,
5′-GCGGCCTTAGCATTAGGAGT-3′,
reverse,
5′-TTGGAAGAGGGGAACCCGTA-3′;
MSX1,
forward,
5′-TTGCCACTCGGTGTCAAAGT-3′,
reverse,
5′-AAGGGGACACTTTGGGCTTG-3′;
GAPDH,
forward,
5′-GAAAGCCTGCCGGTGACTAA-3′,
reverse,
5′-GCCCAATACGACCAAATCAGAG-3′.

To determine the purified DNA from ChIP samples, quantitative PCR was performed with RT² SYBR Green/ROX PCR Master Mix and the PCR reactions were examined by 2% agarose gel electrophoresis. PCR primers were designed to cover the canonical TCF/β-catenin binding motifs of 6 randomly chosen β-catenin target genes (shown in FIG. 4c). The sequences and genomic locations (NCBI36/hg18) of PCR primers are listed below. GAPDH was used as a negative control.

| | | Genomic | Amplicon |
|---|---|---|---|
| Gene | Primer sequence | location | size (bp) |
| AXIN2 | Forward: | Chr17: | 118 |
| | 5′-GGCTTTCTTTGA | 60987984- | |
| | AGCGGCTC-3′ | 60988003 | |
| | Reverse: | Chr17: | |
| | 5′-TAACCCCTCAGA | 60988082- | |
| | GCGATGGA-3′ | 60988101 | |
| FGF19 | Forward: | Chr11: | 50 |
| | 5′-GGTTCTTGGCTG | 69228272- | |
| | GGAGAGTT-3′ | 69228291 | |
| | Reverse: | Chr11: | |
| | 5′-GCCCCAGTTGTC | 69228302- | |
| | GATTGCTT-3′ | 69228321 | |
| MSX1 | Forward: | Chr4: | 61 |
| | 5′-GCTGGCGCCTTA | 4909572- | |
| | AAACAACA-3′ | 4909592 | |
| | Reverse: | Chr4: | |
| | 5′-TGTGTCACGATT | 4909613- | |
| | CCTCAGCG-3′ | 4909632 | |
| Wnt6 | Forward: | Chr2: | 80 |
| | 5′-GAGGACGACCCA | 219434244- | |
| | GGTCCTAT-3′ | 219434263 | |
| | Reverse: | Chr2: | |
| | 5′-TCTGCTTCAAAG | 219434304- | |
| | CTGCCACT-3′ | 219434323 | |
| IFI27 | Forward: | Chr14: | 112 |
| | 5′-CAGAGGCCATTT | 93651214- | |
| | CCCCTTGG-3′ | 93651234 | |
| | Reverse: | Chr14: | |
| | 5′-CTGAATGGGCAT | 93651306- | |
| | TACCAGCC-3′ | 93651325 | |
| MYH7B | Forward: | Chr20: | 123 |
| | 5′-GAGTCCTTCTTC | 33023132- | |
| | CTGCCACC-3′ | 33023151 | |
| | Reverse: | Chr20: | |
| | 5′-TCAAAGAGCTCT | 33023235- | |
| | GAGCGACG-3′ | 33023254 | |
| GAPDH | Forward: | Chr12: | 52 |
| | 5′-CGCTCACTGTTC | 6514210- | |
| | TCTCCCTC-3′ | 6514229 | |
| | Reverse: | Chr12: | |
| | 5′-ATGGTGTCTGAG | 6514242- | |
| | CGATGTGG-3′ | 6514261 | |

Statistical Analysis

For mice and adult zebrafish experiments, no statistical methods were used to predetermine sample size but animals in each group were selected randomly. The sample size (n) of each experiment is indicated in the relevant figure legends. All experiments using culture cells and zebrafish embryo were repeated at least with three biological replicates. A two-tailed Student's t-test was performed to evaluate the statistical difference for all pairwise comparisons. Fisher's exact test was used to analyze the proportions or calculate the probability of overlap between gene lists. Pooled data are represented by the mean and error bars (s.d.) of the replicated experiments. P values are indicated in figure legends and significant differences were considered when *P≤0.05 and **P≤0.01. To our observation, all the measured data is normally distributed and the variance is similar between the groups that are being statistically compared.

Results and Discussion

In human cancers most CTNNB1 and APC mutations lead to failure of GSK3β phosphorylation and β-TrCP ubiquitination of β-catenin, both result in its nuclear accumulation and aberrant activation of Wnt signaling. In a reporter based screening of FDA approved drugs, axitinib showed the strongest inhibition of Wnt/β-catenin signaling activated by GSK3β inhibitor 6BIO (FIG. 2). Wnt inhibition by axitinib was confirmed in 293FT cells overexpressing β-catenin-4A (Ser33A/Ser37A/Thr41A/Ser45A) with mutant GSK3β phosphorylation sites (FIG. 3). In zebrafish embryos, GSK3β inhibition enhances Wnt signaling and generates an eyeless phenotype, which was dose-dependently and totally rescued by axitinib (FIG. 3c). In transgenic zebrafish carrying a TCF-GFP reporter, axitinib decreased TCF-GFP expression in the midbrain/hindbrain boundary and fin mesenchyme where Wnt signals are required during development (FIG. 3d). Collectively, these results demonstrate that axitinib inhibits Wnt/β-catenin signaling in vitro and in vivo.

We next examined axitinib in APC mutant cancer cells and tumors. SW480 cells show the highest Wnt/β-catenin signaling activity in a profiling of 85 cancer cell lines, flow cytometry confirmed 98% cells with activated Wnt signaling based on the Wnt reporter 7TGC (7×TCF-GFP-SV40-mCherry) (data not shown). Axitinib treatment increased TCF-GFP^{low} cells and induced apoptosis in these cells, implying the Wnt dependent viability of SW480 cells (FIG. 3e).

In soft agar assay, colonies formed in SW480 (Wnt activated) and HCT116 (Wnt activated) cells but not in RKO cells (Wnt inactivated), suggesting that colony formation of colon cancer cells is dependent on Wnt/β-catenin signaling (FIG. 3h). Significantly, axitinib strongly and dose-dependently inhibited colony growth of both SW480 and HCT116 cells (FIG. 3i).

In Apc^{min/+} mice adenomas are initiated due to mutant Apc and marked by increase of nuclear β-catenin. Treatment with axitinib at 50 mg/kg daily for 5 weeks significantly decreased the numbers of both multi-villus adenomas and microadenomas (FIG. 3f). Obviously weaker staining of the proliferation marker Ki67 in axitinib treated adenomas further supports the inhibition of oncogenic Wnt signaling by axitinib (FIG. 3g).

Cancer tissue-derived organoids recapitulate the cellular heterogeneity of tumors and represent an attractive preclinical model for precise evaluation of anti-cancer drugs under physiologically relevant conditions. In contrast to wildtype intestinal organoids requiring external Wnt signal activation (e.g. R-spondin), Apc deleted organoids exhibit β-catenin activation independent of R-spondin. We tested axitinib in Apc-deleted murine organoids and axitinib strongly inhibited their growth (FIG. 3j).

Wnt/β-catenin signaling is required for maintaining the intestinal crypt integrity and homeostasis. Unexpectedly, no obvious change was found in the normal-looking small intestine of axitinib treated mice in terms of mucosa organization, crypt density and the distribution of Ki67 positive cells (data not shown). To further characterize the normal Wnt dependent process, we assessed axitinib in adult zebrafish with resected tailfins where Wnt/β-catenin signaling is activated and required for tissue regeneration. Indeed, the tailfin regrowth and TCF-GFP activation were completely inhibited in all fish treated with axitinib for 6 consecutive days (FIG. 3h). However, no significant change was found in the intestine of axitinib treated fish based on the crypt-villus architecture and incorporation of the mitotic marker BrdU (data not shown). Taken together, axitinib inhibits Wnt signaling in tumor growth and tissue regeneration with minor effect on adult tissue homeostasis.

We hypothesized that axitinib affects cell division considering that it is largely symmetric in tumor and resected tailfin for uncontrolled growth or tissue repair, whereas it is mainly asymmetric in normal intestine to maintain tissue homeostasis. Indeed, imaging of axitinib treated SW480-7TGC cells revealed up to 10% paired cells with unequal TCF-GFP expression while this was never found in control cells (FIG. 4a). The switch from symmetric cell division (SCD) to asymmetric cell division (ACD) in terms of Wnt signaling activity (Wnt ACD) was confirmed by live cell imaging and prostate EPT3-7TGC cells treated with 6B10, as well as SW480 cells containing a 7×TCF-mCherry (7TC) reporter (data not shown). Consistent with the induction of apoptosis in TCF-GFP^{low} cells (FIG. 3e), less frequent Ki67 staining in TCF-mCherry^{low} cells supports the Wnt-dependent proliferation of SW480 cells (FIG. 4b).

The observation of Wnt ACD encouraged us to assess non-random DNA segregation that is a well-established ACD feature of stem cells. In the EdU (5-ethynyl-2′-deoxyuridine) label release assay, only the newly synthesized DNA will be labeled with EdU during cell division. Dramatically, around 15% of the axitinib treated SW480 cells exhibited unequal EdU labelling (EdU ACD), which was never found in control cells (FIG. 4c). In HCT116 and RKO colon cancer cells with activated and silenced Wnt signaling, respectively, EdU ACD was absent in both cell types, but increased significantly in axitinib treated HCT116 cells (FIG. 4d). In RKO cells, pre-treatment with 6B10 was required for EdU ACD induction by axitinib (FIG. 4d), indicating that axitinib induced non-random DNA segregation is dependent on the inhibition of Wnt signaling.

Axitinib induced ACD was further supported by immunofluorescence staining of β-catenin, nearly 19% of axitinib treated cells displayed unequal distribution of nuclear β-catenin (β-catenin ACD) (FIG. 4e). To ask for the association among EdU, Wnt and β-catenin ACD, we performed correlation analysis of EdU ACD and Wnt ACD in SW480-7TC cells. Daughter cells with higher Wnt signaling preferably inherited the EdU unlabeled DNA (FIG. 4f). Consistently, β-catenin higher cells showed exclusively stronger Wnt activity and frequently negative EdU staining (FIGS. 4g and 4h). Given that loss of ACD is critical for tumor initiation and progression, re-establishment of ACD in cancer cells by axitinib could potentially inhibit the malignancy.

The observed β-catenin ACD raises the possibility that axitinib depletes nuclear β-catenin. Western blots confirmed the dose and time dependent decrease of β-catenin in axitinib treated SW480 cells (FIG. 5a). In the presence of proteasome inhibitor MG132, axitinib failed to reduce β-catenin but increased its ubiquitination (FIG. 5b). Additional treatment with the nuclear export inhibitor leptomycin B demonstrated the nuclear location of β-catenin degradation (FIG. 5c), which is supported by the reduced nuclear β-catenin in axitinib treated adenomas (FIG. 5d). Expectedly, knockdown of β-catenin readily induced Wnt and EdU ACD (FIG. 5e), indicating that axitinib directs ACD by promoting nuclear β-catenin degradation.

The N-terminal residues Ser45/Thr41/Ser37/Ser33 of β-catenin are required for GSK3β phosphorylation and β-TrCP ubiquitination in the presence of APC and predominantly mutated in cancer patients. In APC mutant SW480 cells axitinib reduced both phosphorylated and non-phospho (active) β-catenin (ABC), as well as β-catenin-4A (FIGS. 5f and 5g). Independent of GSK3β, APC and β-TrCP, β-catenin can be degraded by TRIM33 targeting Ser715 or autophagy protein LC3 targeting W504/1507, both possibilities were ruled out since axitinib reduced β-catenin with mutations of all these residues (FIG. 5g), indicating an undescribed mechanism in axitinib induced β-catenin degradation.

Axitinib is a known inhibitor of vascular endothelial growth factor receptors (VEGFRs), which was confirmed by in vitro kinase activity assay (FIG. 7a). However, the expression of VEGFRs and other kinase targets of axitinib (FLT1, KDR, FLT4, KIT, FLT3, PDGFRA, PDGFRB) is very low in SW480 cells (FIGS. 7b, 7c and 7f) and other VEGFR inhibitors did not show inhibition of Wnt/β-catenin signaling (FIGS. 7d and 7e). Importantly, loss of VEGFR1 does not reduce β-catenin and VEGFR inhibitors block the regenerative angiogenesis but not tailfin regrowth in zebrafish, suggesting that axitinib inhibits Wnt signaling independent of VEGFRs in vitro and in vivo.

To identify the target proteins of axitinib, we used a strategy called DARTS (drug affinity responsive target stability) according to which target proteins are assumed to be protected from proteolysis by binding to small molecules. We combined DARTS to proteomic approaches and identified a number of proteins strikingly protected by axitinib (FIG. 6a). The most frequently detected protein MED23 is a subunit of the mediator complex that functions as a bridge between transcription activators and RNA polymerase II. Interestingly, one detected protein SHPRH is an E3 ubiquitin ligase in the nucleus. The direct binding of MED23 and SHPRH to axitinib in intact living cells was confirmed by the cellular thermal shift assay (CETSA) (FIG. 6b).

Independent evaluation of the direct binding of axitinib and SHPRH was performed by microscale thermophoresis (MST), an all-optical approach measuring the directed motion of GFP-tagged proteinsin temperature gradients. SW480 cell lysates containing GFP-tagged SHPRH or free GFP as a control were incubated with axitinib briefly (less than 1 min) before MST assay. As expected, a robust binding curve was observed in the GFP-SHPRH sample with a Kd at 10.4±3.3 μM (FIG. 6j). Of note, in another experiment the Kd value of human interferon gamma and an antibody thereto determined by MST was 16 times higher in cell lysates than PBS buffer, implying that the Kd of axitinib and purified SHPRH could be at the nanomole level.

In addition, chromatin immunoprecipitation (ChIP) confirmed the occupancy of MED23 at the consensus TCF/LEF-binding motifs of Wnt target genes. Treatment with axitinib reduced MED23 occupancy and repressed target genes (FIG. 6c). Reciprocal co-immunoprecipitation (CoIP) demonstrated the endogenous association of MED23 and β-catenin in SW480 cells, and this association was disrupted by axitinib (FIG. 6d).

Given that SHRPH is an E3 ubiquitin ligase, we hypothesized that axitinib binding to SHPRH promotes β-catenin degradation. Indeed, treatment with axitinib increased the stability of SHPRH in presence of protein biosynthesis inhibitor cycloheximide, and decreased its ubiquitination level (FIG. 6e). Similar to axitinib treatment, overexpression of GFP tagged SHPRH in SW480 cells reduced not only endogenous β-catenin, but also β-catenin-4A, β-catenin truncated at N-terminal (ΔN47) or C-terminal (AC) (FIG. 6f). As expected, ubiquitylated β-catenin was increased by overexpression of SHPRH (FIG. 6g). On the other hand, knockdown of SHPRH blocked the decrease of β-catenin by axitinib (FIG. 6h).

Our results demonstrate that axitinib blocks Wnt/β-catenin signaling by binding of MED23 and SHPRH to dissociate β-catenin from the Mediator and deplete nuclear β-catenin, respectively (FIG. 6i). Given that SHPRH is a chromatin binding ubiquitin ligase and Mediator is the ultimate functional target for DNA-binding transcription factors, axitinib blocks Wnt/β-catenin signaling at the endpoint of the pathway and would be effective against oncogenic Wnt/β-catenin signaling independent of the mutant genes and as such would also be effective against other diseases or conditions in which WNT/β-catenin signal transduction is a contributing factor.

### Example 2—Pazopanib, Orlistat and Topotecan Block Wnt/β-Catenin Signaling in a Human Embryonal Kidney Cell Line

TOPFlash assay was performed as described above. Briefly, for each well of a 96-well plate 293FT cells were transfected with 0.22 μg TOPFlash (or FOPFlash with mutant TCF binding sites) and 0.02 μg pRL-SV40P using lipofectamine 3000. For co-transfection with other plasmids, 0.1 μg additional DNA was used. Four hours after transfection, 293FT cells were treated with DMSO, 1 μM 6B10 and 1 μM 6B10 together with pazopanib, orlistat and topotecan at varying concentrations for 24 hours. The luciferase activity was then measured using Dual-Glo® Luciferase Assay System (Promega, E2940) according to the recommended protocol. The TOPFlash or FOPFlash activity was normalized to Renilla luciferase signals. Results are shown in FIG. 8

1. A method for the treatment or prevention of a disease or condition in which WNT/β-catenin signal transduction is a contributing factor, said method comprising administering to a subject in need thereof one or more WNT/β-catenin signal transduction inhibitors selected from axitinib,
(ii) pazopanib,
(iii) orlistat,
(iv) topotecan,
(v) pharmaceutically effective substitution derivatives thereof wherein one or more hydrogen groups are substituted with SR¹ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, and
(vi) pharmaceutically acceptable salts, or solvates or hydrates thereof, diastereoisomers, tautomers, enantiomers, and prodrugs and active metabolites thereof.

2. (canceled)

3. (canceled)

4. (canceled)

5. (canceled)

6. The method of claim 1, wherein said method is for the treatment or prevention of a hyperproliferative or neoplastic disease or condition in which WNT/β-catenin signal transduction in a neoplastic cell is a contributing factor.

7. The method of claim 6, wherein said hyperproliferative or neoplastic disease or condition is a cancer or a malignant or premalignant tumour.

8. The method of claim 1, wherein said WNT/β-catenin signal transduction is WNT/β-catenin oncogenic signalling.

9. The method of claim 7, wherein said cancer or a malignant or premalignant tumour carries one or more agonistic mutant forms of the components of the WNT/β-catenin signal transduction pathway.

10. The method of claim 9, wherein said agonistic mutant form of the components of the WNT/β-catenin signal transduction pathway is selected gain of function mutations in one or more of a CTNNB1, WNT, FZD or TCF gene and loss of function mutations in one or more of an APC or an Axin gene.

11. The method of claim 1, wherein said method is for the treatment or prevention of a hyperproliferative or neoplastic disease or condition in which WNT/β-catenin signal transduction in an immune cell is a contributing factor.

12. (canceled)

13. The method of claim 1, wherein said disease or condition may be selected from colorectal cancer, prostate cancer, testicular cancer, skin cancer, breast cancer, kidney cancer, ovarian cancer, stomach cancer, intestinal cancer, liver cancer, pancreatic cancer, lung cancer, oesophageal cancer, oral cancer, throat cancer, brain cancer, adrenal cancer, thyroid cancer, uterine cancer, haematological cancer, colorectal polyps, pilomatrixoma, hemangioma, osteoma, chondroma, lipoma, fibroma, lymphangioma, leiomyoma, rhabdomyoma, astrocytoma, meningioma, ganglioneuroma, papilloma, adenoma.

14. (canceled)

15. The method of claim 11, wherein said method is for combating cancer mediated immune suppression and/or to augment anti-cancer immunity.

16. The method of claim 1, wherein said method is for the treatment or prevention of an immune or inflammatory disease or condition in which WNT/β-catenin signal transduction is a contributing factor.

17. The method of claim 11, wherein said WNT/β-catenin signal transduction is in an immune cell selected from monocytes, macrophages, neutrophils, DCs, mast cells, natural killer cells, T cells and B cells, eosinophils and basophils.

18. (canceled)

19. The method of claim 1, wherein said disease or condition is selected from an autoimmune disease, inflammatory bowel disease, colitis, atherosclerosis, neurodegeneration, neuroinflammation, allograft organ transplant rejection, allergy, a chronic microbial infection or a chronic wound.

20. (canceled)

21. The method of claim 1, wherein said disease or condition is a disorder or dysfunction in the metabolism of carbohydrates by a subject.

22. The method of claim 21, wherein said disorder or dysfunction in the metabolism of carbohydrates by a subject is diabetes mellitus type 2, insulin resistance, metabolic syndrome, obesity and diabetic retinopathy, nephropathy and neuropathy.

23. (canceled)

24. The method of claim 1 wherein said method is to promote the healing of a chronic wound.

25. The method of claim 1, wherein said disease or condition is a high bone mass disorder or sclerosteosis.

26. The method of claim 1, wherein said WNT/β-catenin signal transduction inhibitor is used together with a further pharmaceutical for the treatment of said disease or condition in which WNT/β-catenin signal transduction is a contributing factor, wherein said further pharmaceutical is selected from a cytotoxic chemotherapy agent, an angiogenesis inhibitor, an anti-cancer monoclonal antibody, a radioimmunotherapeutic, a cancer treatment vaccine, an immunostimulatory agent, an immunosuppressant, a corticosteroid, a non-steroidal anti-inflammatory drug (NSAID), an antibiotic, an antifungal, an antiviral, an oral antidiabetic drug or an injectable antidiabetic drug.

27. (canceled)

28. A method for the immunotherapy of a hyperproliferative or neoplastic disease or condition in a subject in which DCs are administered to the subject, said method comprising administering an effective amount of one or more of the WNT/β-catenin signal transduction inhibitors selected from axitinib,
(ii) pazopanib,
(iii) orlistat,
(iv) topotecan,
(v) pharmaceutically effective substitution derivatives thereof wherein one or more hydrogen groups are substituted with SR¹ (wherein R¹═H or C₁₋₃ alkyl, e.g. —CH₃), NR₂ (wherein R₂ is independently H or C₁₋₃ alkyl, e.g. —CH₃), Cl, Br, NO₂ and OH, and
(vi) pharmaceutically acceptable salts, or solvates or hydrates thereof, diastereoisomers, tautomers, enantiomers, and prodrugs and active metabolites thereof,
to a subject at the same time as, or substantially the same time as, or prior to, or after said subject receives the DCs.

29. The method of claim 28, wherein said method comprises providing a sample of DCs in vitro, and either
(a) administering said DCs to the subject at the same time as, or substantially the same time as, or prior to, or after an effective amount of said WNT/β-catenin signal transduction inhibitor; or
(b1) contacting said DCs with an effective amount of one or more of said WNT/β-catenin signal transduction inhibitor, and
(b2) administering the inhibitor-treated DCs to the subject.

30. The method of claim 28, wherein the DCs are administered in an immature form, and/or

31. The method of claim 28, wherein the DCs are administered intratumorally or to the site of a tumour following ablation or other such destruction of the tumour.

32. An in vitro method for diagnosing WNT/β-catenin dependent cancers, said method comprising
(i) contacting a sample of cells from a test cancer with one or more of the WNT/β-catenin signal transduction inhibitors selected from
(a) axitinib,
(b) pazopanib,
(c) orlistat,
(d) topotecan,
(e) pharmaceutically effective substitution derivatives thereof wherein one or more hydrogen groups are substituted with SR¹ (wherein R¹═H or C₁₋₃ alkyl, e.g. —CH₃), NR₂ (wherein R is independently H or C₁₋₃ alkyl, e.g. —CH₃), Cl, Br, NO₂ and OH, and
(f) pharmaceutically acceptable salts, or solvates or hydrates thereof, diastereoisomers, tautomers, enantiomers, and prodrugs and active metabolites thereof, and
(ii) assessing the effects of said inhibitor on said sample.

33. The method of claim 16, wherein said WNT/β-catenin signal transduction is in an immune cell selected from monocytes, macrophages, neutrophils, DCs, mast cells, natural killer cells, T cells and B cells, eosinophils and basophils.


SEQUENCE LISTING (32 sequences)

SEQ ID NO: 1 - DNA, 36 bp, Artificial Sequence
  Oligonucleotide primer
ccttgctcaccatggatcctttaccaacagtaccgg

SEQ ID NO: 2 - DNA, 43 bp, Artificial Sequence
  Oligonucleotide Primer
cgcccttgctcaccatggatccctttttgcaaaagcctaggcc

SEQ ID NO: 3 - DNA, 44 bp, Artificial Sequence
  Oligonucleotide Primer
gtctgaggacaagccataagattacaagaaacggctttcagttg

SEQ ID NO: 4 - DNA, 45 bp, Artificial Sequence
  Oligonucleotide Primer
tggatatcgccaggatgatcctgcagatcgttcttttcactctgg

SEQ ID NO: 5 - DNA, 21 bp, Artificial Sequence
  Oligonucleotide Primer
cgcacagtttgctggagatca

SEQ ID NO: 6 - DNA, 21 bp, Artificial Sequence
  Oligonucleotide Primer
cctccgagtactgaagcagcc

SEQ ID NO: 7 - DNA, 21 bp, Artificial Sequence
  Oligonucleotide Primer
gaggaaggagacaggtcgcag

SEQ ID NO: 8 - DNA, 22 bp, Artificial Sequence
  Oligonucleotide Primer
tttgtgctttgggcactatggg

SEQ ID NO: 9 - DNA, 20 bp, Artificial Sequence
  Oligonucleotide Primer
ctgcaacaggacattcggga

SEQ ID NO: 10 - DNA, 20 bp, Artificial Sequence
  Oligonucleotide Primer
cagctcgcccatagaacagg

SEQ ID NO: 11 - DNA, 20 bp, Artificial Sequence
  Oligonucleotide Primer
atcagcagtgaccagtgtgg

SEQ ID NO: 12 - DNA, 20 bp, Artificial Sequence
  Oligonucleotide Primer
tggccacaactcctccaatc

SEQ ID NO: 13 - DNA, 20 bp, Artificial Sequence
  Oligonucleotide Primer
gcggccttagcattaggagt

SEQ ID NO: 14 - DNA, 20 bp, Artificial Sequence
  Oligonucleotide Primer
ttggaagaggggaacccgta

SEQ ID NO: 15 - DNA, 20 bp, Artificial Sequence
  Oligonucleotide Primer
ttgccactcggtgtcaaagt

SEQ ID NO: 16 - DNA, 20 bp, Artificial Sequence
  Oligonucleotide Primer
aaggggacactttgggcttg

SEQ ID NO: 17 - DNA, 20 bp, Artificial Sequence
  Oligonucleotide Primer
gaaagcctgccggtgactaa

SEQ ID NO: 18 - DNA, 22 bp, Artificial Sequence
  Oligonucleotide Primer
gcccaatacgaccaaatcagag

SEQ ID NO: 19 - DNA, 20 bp, Artificial Sequence
  Oligonucleotide Primer
ggctttctttgaagcggctc

SEQ ID NO: 20 - DNA, 20 bp, Artificial Sequence
  Oligonucleotide Primer
taacccctcagagcgatgga

SEQ ID NO: 21 - DNA, 20 bp, Artificial Sequence
  Oligonucleotide Primer
ggttcttggctgggagagtt

SEQ ID NO: 22 - DNA, 20 bp, Artificial Sequence
  Oligonucleotide Primer
gccccagttgtcgattgctt

SEQ ID NO: 23 - DNA, 20 bp, Artificial Sequence
  Oligonucleotide Primer
gctggcgccttaaaacaaca

SEQ ID NO: 24 - DNA, 20 bp, Artificial Sequence
  Oligonucleotide Primer
tgtgtcacgattcctcagcg

SEQ ID NO: 25 - DNA, 20 bp, Artificial Sequence
  Oligonucleotide Primer
gaggacgacccaggtcctat

SEQ ID NO: 26 - DNA, 20 bp, Artificial Sequence
  Oligonucleotide Primer
tctgcttcaaagctgccact

SEQ ID NO: 27 - DNA, 20 bp, Artificial Sequence
  Oligonucleotide Primer
cagaggccatttccccttgg

SEQ ID NO: 28 - DNA, 20 bp, Artificial Sequence
  Oligonucleotide Primer
ctgaatgggcattaccagcc

SEQ ID NO: 29 - DNA, 20 bp, Artificial Sequence
  Oligonucleotide Primer
gagtccttcttcctgccacc

SEQ ID NO: 30 - DNA, 20 bp, Artificial Sequence
  Oligonucleotide Primer
tcaaagagctctgagcgacg

SEQ ID NO: 31 - DNA, 20 bp, Artificial Sequence
  Oligonucleotide Primer
cgctcactgttctctccctc

SEQ ID NO: 32 - DNA, 20 bp, Artificial Sequence
  Oligonucleotide Primer
atggtgtctgagcgatgtgg